(19) United States
(12) Patent Application Publication
(10) Pub. No.: US 2008/0213853 A1
(43) Pub. Date: Sep. 4, 2008

(54) Magnetofluidics

(76) Inventors: Antonio Garcia, Chandler, AZ (US); Solitaire Lindsay, Phoenix, AZ (US); Mark A. Hayes, Gilbert, AZ (US); John Devens Gust, Mesa, AZ (US)

Correspondence Address: FISH & RICHARDSON, PC, P.O. BOX 1022, MINNEAPOLIS, MN, 55440-1022, US

(21) Appl. No.: 11/952,088

(22) Filed: Dec. 6, 2007

Related U.S. Application Data
(63) Continuation-in-part of application No. PCT/US07/62842, filed on Feb. 27, 2007.
(60) Provisional application No. 60/868,892, filed on Dec. 6, 2006.
(60) Provisional application No. 60/777,679, filed on Feb. 27, 2006.

(51) Int. Cl. C12N 13/00; B01J 19/00; C12M 1/00

(52) U.S. Cl. 435/173.1; 422/291; 435/283.1

(57) ABSTRACT

Magnetofluidic systems and techniques. In one aspect, a magnetofluidic device includes a superhydrophobic surface and a fluid sample in physical contact with the superhydrophobic surface, the fluid sample comprising a collection of particles coated with a passivating layer. The particles are magnetically active in that they respond to an applied magnetic field.

### CROSS-REFERENCE TO RELATED APPLICATIONS

This application is a continuation-in-part and claims the priorities of U.S. Provisional Application Ser. No. 60/868,892, filed on Dec. 6, 2006 and International Application No. PCT/U.S. 07/62842 filed on Feb. 27, 2007, which in turn claims the priority of U.S. Provisional Application Ser. No. 60/777,679 filed on Feb. 27, 2006. The contents of all of all three of these applications are incorporated herein by reference.

### GOVERNMENT FUNDING

At least some of the technology described herein was made with government support from the National Science Foundation under the following grant numbers: HRD-0450137 & HRD-9978868. The United States Government may have certain rights in the invention.

### BACKGROUND

Controlling droplet movement under the influence of a stimulus is a capability of continued and growing interest. Although drop dynamic behavior on a superhydrophobic surface is interesting from a scientific and technologic point of view, little is known about aqueous drops moving on a flat non-patterned superhydrophobic surface by mechanisms different from gravity. There are several examples of technologies that can benefit from key advances in this field, such as superhydrophobic surfaces capable of self-cleaning by the action of a rolling drop or microfluidics devices that take advantage of new effects and better performance derived from manipulating fluids at small scales. For further discussion, see Quéré, D., Fakir droplets. Nature Materials, 2002. 1: p. 14-15; Gould, P., Smart, clean surfaces. Materials Today, 2003. 6(11): p. 44-48; Nguyen, N.-T. and S. T. Wereley, Fundamentals and applications of microfluidics. 2002, Norwood, Mass.: Artech House, all of which are herein incorporated by reference. A number of fascinating phenomena have been reported in the literature treating the dynamic behavior of non-wetting drops. Most of them focused on drop dynamics on patterned non-wetting surfaces. Examples of these are studies on the dynamics of drops rolling over an inclined superhydrophobic surface through the action of gravity or by spreading on a flat, patterned superhydrophobic surface. For further discussion, see Quéré, D. and D. Richard, Viscous drops rolling on a tilted non-wettable solid. Europhysics letters, 1999. 48(3): p. 286-291; Mahadevan, L. and Y. Pomeau, Rolling droplets. Physics of fluids, 1999. 11(9): p. 2449-2453; McHale, G., et al., Topographydriven spreading. Physical review letters, 2004. 93(3), all of which are herein incorporated by reference. Relaxation and contact line dynamics have been studied in drops generated by drop-wise condensation on superhydrophobic geometrically patterned surfaces that grow until they become large enough to touch and coalesce. For further discussion, see Beysens, D., Phase transition, contact line dynamics and drop coalescence, in International workshop on dynamics of complex fluids. 2004, Yukawa Institute at Kyoto University: Kyoto, Japan, which is herein incorporated by reference. Other studies used a water drop placed between a hydrophilic and a superhydrophobic patterned surface in order to measure fluid pressure (water) effects on contact angle. For further discussion, see Journet, C., et al., Carbon angle measurements on superhydrophobic carbon nanotube forests: effect of fluid pressure. Europhysics letters, 2005. 71(1): p. 104-109, which is herein incorporated by reference. Also, the contact angle of a drop on a superhydrophobic surface can be modified using light. For further discussion, see Rosario, R., et al., Lotus Effect Amplifies Light-Induced Contact Angle Switching. J. Phys. Chem. B, 2004. 108: p. 12640-12642, which is herein incorporated by reference.

Digital microfluidics is an alternative paradigm for manipulation of discrete droplets, where processing is performed on unit-sized packets of fluid which are transported, stored, mixed, reacted, or analyzed in a discrete manner. This concept can be demonstrated using electrowetting arrays for droplet transportation without the use of pumps or valves. For further discussion, see Duke University, Digital Microfluidics by Electrowetting (Jun. 7, 2004), http://www.ee.duke.edu/research/microfluidics/, which is herein incorporated as Appendix A; and see also Srinivasan, V., V. K. Pamula, and R. B. Fair, proplet-based microfluidic lab-on-a-chip for glucose detection. Analytica Chimica Acta, 2004. 507(1): p. 145-150; Ren, H., et al., Dynamics of electro-wetting droplet transport. Sensors and Actuators B: Chemical, 2002. 87(1): p. 201-206, both of which are herein incorporated by reference.

The physics of scale require that microfluidic devices exploit new approaches to fluid movement because of an inherently large ratio of liquid surface area to volume. One promising method is the control of movement of fluid droplets by magnetic fields. Magnetic fields can be easily imposed by permanent or electromagnets, can be accurately controlled, and are typically mild enough to pose no danger to biological materials. However, moving water-based droplets with magnetic fields has not been effectively demonstrated previously because, inter alia, the droplet movement is retarded by the typically low contact angle between the droplet and the surface.

Therefore, there remains a need for methods and compositions that overcome these deficiencies and that effectively provide for digital microfluidic methods with the driving force being the use of magnetic fields.

Oxidation corrosion or chemical corrosion of magnetic particles used in technical systems harms the stability and application of these particles. For further discussion, see American Institute of Physics Handbook 5:144-51 (Dwight E. Gray, Ph.D. et al, eds., 1972); Park, J. H., Chin, B. D., and Park O. O., J. Colloid Interface Sci. 240: 349-354 (2001), both of which are herein incorporated by reference. Researchers have identified techniques for improving the chemical stability of soft magnetic particles exposed to oxidizing magnetorheological fluids. See, for example, J. C. Ulicny, A. M. Mance, Mater. Sci Eng. A 369:309-313 (2004); U.S. Pat. No. 5,382,373 (filed Oct. 30, 1992); U.S. Pat. No. 5,505,880 (filed Nov. 16, 1994); M. S. Cho, S. T. Lim, I. B. Jang, H. J. Choi, M. S. Jhon, IEEE Trans. Magn., 40(4):3036-3038 (2004); U.S. Pat. No. 6,787,058 (filed Nov. 12, 2002), all of which are herein incorporated by reference. Specifically, several studies have described encapsulating magnetic particles with polysiloxane to improve their chemical stability in oxidizing magnetorheological fluids. H. T. Pu, F. J. Jiang, and Z. L. Yang, Mat. Lett. 60:94-97 (2006); U.S. patent application Ser. No. 10/128,573 (filed Apr. 24, 2002), both of which are herein incorporated by reference. In a 2006 study by Pu et al., for example, the researchers prepared a soft magnetic composite of reduced iron particles encapsulated with polysiloxane nanofilm shell by hydrolysis-condensation polymerization of tetraethylorthosilicate (TEOS) on the iron particles' surfaces. The group observed that encapsulation with polysiloxane nanofilm greatly improved both the iron particles' resistance to thermal oxidation and their resistance to corrosion by acids. They concluded that encapsulated iron particles were useful in magnetorheological fluids because the particles' magnetic properties, although compromised, were still much better than other untreated soft magnetic particles that are less susceptible to corrosion, such as Fe₃O₄.

### SUMMARY

This disclosure relates to magnetofluidics, including magnetofluidic systems and techniques, along with methods of manufacturing, performing, and operating such systems and techniques.

In one aspect, a magnetofluidic device includes a superhydrophobic surface and a fluid sample in physical contact with the superhydrophobic surface. The fluid sample includes a collection of particles coated with a passivating layer. The particles are magnetically active in that they respond to an applied magnetic field.

This and other aspects can include one or more of the following features. The passivating layer can include a silicon-oxygen backbone. The passivating layer can include a polysiloxane. The passivating layer can include a polymerization product of tetraorthosilicate. The magnetofluidic device can include a digital magnetofluidic device.

The fluid sample can be an aqueous solution. The fluid sample can include a bodily fluid. The fluid sample can include a solution of a biologically active agent, a pharmaceutically active agent, or a mixture thereof. The collection of particles can include a collection of paramagnetic particles. The superhydrophobic surface can include a nanostrutured surface. The magnetofluidic device can also include a device to apply a magnetic field to the fluid sample, wherein the applied magnetic field is of sufficient strength to induce movement of the fluid sample.

The fluid sample can include a fluid droplet. The passivating layer can include a hydrophilic surface. The passivating layer can include a barrier to oxidation of the magnetically active particles and/or a barrier to reaction of the magnetically active particles with the fluid sample. The fluid sample can rest on the superhydrophobic surface.

In another aspect, a method includes applying a magnetic field to a fluid sample that is in contact with a superhydrophobic surface. The fluid sample can include a collection of particles coated with a passivating layer. The particles can be magnetically active in that they respond to the applied magnetic field. The response of the magnetically active particles can either induce or prevent movement of the fluid sample.

This and other aspects can include one or more of the following features. The application of the magnetic field can include moving a source of the magnetic field to induce corresponding movement of the fluid sample. The application of the magnetic field can include changing a magnitude of the applied magnetic field to induce movement of the fluid sample. The application of the magnetic field can include moving the fluid sample into contact with a second fluid sample. The second fluid sample can be pinned at a defect in the superhydrophobic surface. The second fluid sample need not include magnetically active particles.

The method can also include moving a combination of the fluid sample and the second fluid sample. The application of the magnetic field can include splitting the fluid sample into a first aliquot and a second aliquot. The splitting of the fluid sample can include applying an electric field to the fluid sample. The application of the electric field can include isoelectric focusing of a biopolymer in the fluid sample.

In another aspect, a system includes a superhydrophobic surface, a magnetic field source disposed to apply a magnetic field across the superhydrophobic surface, and a fluid sample in physical contact with the superhydrophobic surface, the fluid sample comprising a collection of particles coated with a passivating layer. The particles are magnetically active in that they respond to the applied magnetic field.

This and other aspects can include one or more of the following features. The system can also include a controller to control a magnitude or a direction of the magnetic field. For example, the controller can be configured to change the magnetic field generated by an electromagnet.

The systems and techniques described herein can be used to obtain one or more of the following advantages. Magnetofluidic devices in which encapsulated magnetic particles are included in one or more discrete fluid droplets can be formed. The encapsulated magnetic particles can resist oxidation and/or corrosion. Fluid droplets that include encapsulated magnetic particles can be moved. Movement can be facilitated by placement of the fluid droplets on a superhydrophobic surface and/or by applying and varying a magnetic field and/or an electric field to the one or more fluid droplets. Discrete fluid droplets, including one more fluid droplets that include encapsulated magnetic particles, can be combined. Discrete fluid droplets, including one more fluid droplets that include encapsulated magnetic particles, can be fixed to a particular location in a digital magnetofluidic device. Discrete fluid droplets, including one more fluid droplets that include encapsulated magnetic particles, can be released t from a receptacle into a magnetofluidic device. Discrete fluid droplets, including one more fluid droplets that include encapsulated magnetic particles, can be cleaved in a magnetofluidic device. A discrete fluid droplet, including a fluid droplet that includes encapsulated magnetic particles, that includes two or more proteins included can be separated into multiple droplets that each have different concentrations of the two or more proteins.

Additional advantages will be set forth in part in the description which follows, and in part will be obvious from the description, or may be learned by practice. Other advantages will be realized and attained through the elements and combinations particularly pointed out in the appended claims. It is to be understood that both the foregoing general description and the following detailed description are exemplary and explanatory only and are not restrictive of the invention, as claimed.

The aspects and applications of the invention presented here are described below in the drawings and detailed specification. Unless specifically noted, it is intended that the words and phrases in the specification and the claims be given the plain, ordinary, and accustomed meaning to those of ordinary skill in the applicable arts. If any other special meaning is intended for any word or phrase, the specification will clearly state and define the special meaning.

The inventors are also aware of the normal precepts of English grammar. Thus, if a noun, term, or phrase is intended to be further characterized or specified or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that the nouns, terms or phrases be given their plain and ordinary English meaning to those skilled in the applicable arts as set forth above.

Likewise, the inventors are fully informed of the standards and application of the special provisions of 35 U.S.C. § 112, paragraph 6. Thus, the use of the words “function,” “means,” or “step” in the Detailed Description or Description of the Drawings or claims is not intended to somewhere indicate a desire to invoke the special provisions of 35 U.S.C. § 112, paragraph 6, to define the invention. To the contrary, if the provisions of 35 U.S.C. 112, paragraph 6, are sought to be invoked to define the invention, the claims will specifically state the exact phrases “means for” or “step for,” and will clearly recite “a function,” without also reciting in such phrases any structure, material, or act in support of the function. Thus, even when the claims recite a “means for” or “step for” performing a defined function, if the claims also recite any structure, material, or act in support of that means or step, or that perform the recited function, then it is the clear intention of the inventors not to invoke the provisions of 35 U.S.C. § 112, paragraph 6. Moreover, even if the provisions of 35 U.S.C. § 112, paragraph 6, are invoked to define the claimed inventions, it is intended that the inventions not be limited only to the specific structure, material, or acts that are described in the preferred embodiments, but in addition, include any and all structures, materials, or acts that perform the claimed function as described in alternative embodiments or forms of the invention, or that are known or later-developed equivalent structures, material, or acts for performing the claimed function.

### BRIEF DESCRIPTION OF THE DRAWINGS

The accompanying figures, which are incorporated in and constitute a part of this specification, illustrate several embodiments and together with the description serve to explain the principles of the invention.

FIG. 1 shows three photographs showing the difference between a hydrophobic and superhydrophobic surface. The left hand side of this silicon substrate contains nanowires while the right hand side does not. The entire surface is covalently coated with a fluorinated hydrocarbon. The water drops from the needle adhere to the right hand side of the sample while they slide off the left hand side of the sample.

FIG. 2 shows an example of how the selection of a particular rough surface can increase the light-induced contact angle change.

FIG. 3 shows a drop of liquid sitting on a fractally rough composite surface made up of solid and air.

FIG. 4 shows experimental contact angles on the rough surface.

FIG. 5 shows a schematic diagram and still image of a water drop containing aligned paramagnetic particle chains and a rare earth magnet. The schematic illustrates a magnetic field line and the effect of geometry on the angle of paramagnetic particle chain alignment. The magnet is moved to the right and the drop slides along the superhydrophobic surface due to the paramagnetic particle chain's action pushing against the surface tension of the drop.

FIG. 6 shows a sequence of three consecutive still images showing a millimeter-size drop with paramagnetic particles sliding on a superhydrophobic surface sample due to the magnetic field of a permanent magnet moving below the surface. The drop is displaced from position in (a) to (c) by the action of the magnet. Pictures were taken at 10 frames per second.

FIG. 7 shows a distribution of paramagnetic particle aggregated inside a moving drop. Five consecutive frames (a) to (e) of a moving drop taken at 10 frames per second show the relatively homogenous and stable distribution of chains sliding with the moving drop.

FIG. 8 shows a sequence of sketches showing a barrier with a meniscus of liquid before applying a magnetic field, after applying a field and pulling the drop through the barrier, and finally after the surface tension causes the drop to snap off leaving a new meniscus caught between the barriers.

FIG. 9 shows a sequence of sketches showing the use of pressure to apply a fluid droplet to a surface defect.

FIG. 10 shows a schematic illustrating proteins contained within a droplet before migration as related to isoelectric point under an applied electric field.

FIG. 11 shows a schematic illustrating proteins contained within a droplet after migration as related to isoelectric point under an applied electric field.

FIG. 12 shows the chemical structure of spiropyrans and their photoresponsive equilibrium.

FIG. 13 shows the chemical structure of dihydroindolizines and their photoresponsive equilibrium.

FIG. 14 shows the chemical structure of dithienylethenes and their photoresponsive equilibrium.

FIG. 15 shows the chemical structure of dihydropyrenes and their photoresponsive equilibrium.

FIG. 16 shows SEM images of nanowires growing on a silicon oxide surface seeded with gold nanodots. After 8 minutes of growth a dense array of randomly oriented, long and thin silicon nanowires with gold caps is evident.

FIG. 17 shows a blood droplet sliding off a superhydrophobic surface.

FIG. 18 shows a urine droplet sliding off a superhydrophobic surface.

FIG. 19 shows a saliva droplet sticking to a superhydrophobic surface.

FIG. 20 shows coalescence of two drops on a superhydrophobic surface sample. (a) A 4 microliter drop containing paramagnetic particles on the right of the figure was displaced by the action of a permanent magnet toward a 6 microliter pure water drop pinned on a surface defect. (b) The two drops coalesce when they become close enough to touch. (c) The combined drop is removed from the surface defect due to the paramagnetic particles and the external magnetic field. Depinning is due to the use of surface tension as a lever and the paramagnetic particles as the fulcrum.

FIG. 21 shows still frames from a movie showing the splitting of a water drop using magnetic fields. a) Two permanent magnets were placed below the drop. b) The stress placed on the drop by moving two magnets away from each other is evident in the distortion of the drop and the partial split seen at the upper part of the drop. c) After the split, the drop volume is about half of what is seen in (a) and (b). The other half of the drop is out of the field of view of the microscope and thus not seen in these still sequences. d) The remaining drop regains spherical shape.

FIG. 22 shows a polished silicon wafer bearing random silicon nanowires with diameters of 20-50 nm prepared by a vapor-liquid-solid technique.

FIG. 23 shows direct comparisons of water contact angles on adjacent polished and nanowire areas.

FIG. 24 shows a SEM image of a magnetically active substance with improved chemical stability. The magnetically active substance 500 is encapsulated with a layer 501.

### DETAILED DESCRIPTION

Disclosed are the components to be used to prepare the compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, acts in methods of making and using the compositions. Thus, if there are a variety of additional acts that can be performed it is understood that each of these additional acts can be performed with any specific embodiment or combination of embodiments of the methods.

It is understood that the compositions disclosed herein have certain purposes. Disclosed herein are certain structural requirements for performing the disclosed purposes, and it is understood that there are a variety of structures that can perform the same purpose that are related to the disclosed structures, and that these structures will typically achieve the same result.

Disclosed are methods of moving and controlling droplets of fluids on superhydrophobic surfaces through the use of magnetic fields. For further discussion, see Egatz-Gomez, A., et al., Discrete magnetic microfluidics, App. Phys. Lett. 89:34106-1-34106-3 (2006) which is herein incorporated by reference and Egatz-Gomez, A., et al., Presentation, Superhydrophobic Nanowire Surfaces for prop Movement Using Magnetic Fields (May 19, 2006). Small water drops (volume 5-20 μL) that contain fractions of paramagnetic particles as low as, for example, 0.1% wt/wt can be moved on a superhydrophobic surface at relatively high speed (7 cm/s) by displacing a permanent magnet. An aqueous drop pinned to a surface defect can be combined with another drop that contains paramagnetic particles thus making it possible to move the newly formed drop. A drop can also be split using two magnetic fields. This new approach to microfluidics has the advantages of faster and more flexible control over drop movement and manipulation.

### A. MICROFLUIDICS

Microfluidic devices are, essentially, tiny, sophisticated devices that can analyze samples or otherwise manipulate fluids and materials at small scales typically below one millimeter in characteristic length. Continuous flow systems have generally been the default approach towards microfluidics such as the so called lab-on-chip bioassay systems. Fluid droplet based microfluidic applications, however, have become increasingly popular because of their ability to enable spatially and temporally resolved chemistries. Typical microfluidic devices can have one or more channels with at least one dimension less than 1 mm and can be used with common fluids including, for example, whole blood samples, bacterial cell suspensions, protein or antibody solutions, and various buffers.

Molecular diffusion coefficients, fluid viscosity, pH, chemical binding coefficients, and enzyme reaction kinetics can be measured by using microfluidic devices. Microfluidic devices can also be used in many applications relating to clinical diagnostics, for example, capillary electrophoresis, isoelectric focusing, immunoassays, flow cytometry, sample injection of proteins for analysis via mass spectrometry, polymerase chain reaction (PCR) amplification, DNA analysis, cell manipulation, cell separation, cell patterning, chemical and materials synthesis, and chemical gradient formation.

For example, in a microfluidic device, the cells, DNA, or proteins that are used to test the candidate drug efficacy can be reduced so that a small amount of a candidate drug can be mixed with its target and the result recorded. This can reduce the time needed to screen all of the drug candidates and can allow as many tests as possible to be run simultaneously. For example, a microfluidic device can require only a single drop of blood for a battery of twenty to thirty tests and can provide nearly immediate results. Microfluidic devices can also help pharmaceutical companies, for example, screen for new drugs by allowing tests to be run on an extremely small scale and in a simultaneous fashion.

The small size and parallel nature of microfluidic devices can create significant advantages. First, because the volume of fluids within these channels is very small, usually only several nanoliters, the amounts of reagents and analytes used are quite small, compared with traditional analysis methods. Second, fabrication techniques used to construct microfluidic devices can be relatively inexpensive and are compatible with elaborate, multiplexed devices and with mass production. Third, microfluidic devices can be fabricated as highly integrated devices for performing a plurality of activities on the same substrate chip.

Fluids are typically driven through microfluidic devices by either pressure driven flow or by electro-osmotic pumping. In pressure driven flow, the fluid can be pushed through the device by using a positive displacement pump, for example, a syringe pump. Pressure driven flow can be both relatively inexpensive and quite reproducible. In electro-osmotic pumping, an electric field can be applied across the microchannels of the microfluidic device. Ions near the surface of the walls of the microchannels move towards the electrode of opposite polarity, resulting in motion of the fluid near the walls and transfers via viscous forces into convective motion of the bulk fluid.

Other pumping devices that can be used with microfluidic devices include, without limitation, mechanical micropumps, such as centrifugal pumps (CD technology), peristaltic pumps, reciprocating pumps, rotary pumps, sonic pumps, ultrasonic pumps, surface acoustic wave (SAW) pumps and nonmechanical micropumps, such as capillary pumps, thermocapillary micropumps, electrocapillary (electrowetting) micropumps, electro-hydro dynamic (EHD) pumps, EHD static pumps (EHD injection pumps), EHD dynamic pumps (traveling or EHD induction pumps), electrokinetic pumps, electro-osmotic pumps, electrophoretic pumps, magneto-hydro dynamic (MHD) pumps, and dielectrophoretic pumps.

Microfluidic devices have a variety of applications including, without limitation, chemical microplants, lab-on-a-chip (LOC) devices, micro total analysis systems (PTAS), microfactories, microseparation systems, and point-of-care (POC) devices.

Chemical microplants are miniaturized chemical plants. A chemical microplant is generally best suited for a distributed processing of materials at the point-of-use. Such distributed processing could avoid central storage and transportation of toxic substances. Another application could be for substances that are needed only in small quantities.

Lab-on-a-chip (LOC) devices are small chips that can contain microfluidic channels narrower than a human hair. These devices take advantage of the properties of liquids and gases to separate and better allow microsensors to analyze their constituent elements.

Micro Total Analysis Systems (μTAS) are miniaturized systems fabricated by the use of micromechanical technology capable of providing total chemical analysis on a microliter scale. The microdevice, fully integrated for example onto a silicon substrate (chip), can perform sample handling, reagent mixing, sample component separation, and analysis. A major area of interest has been the transfer of separation techniques such as capillary electrophoresis (CE) and high performance liquid chromatography (HPLC) to the chip format, coupled with detection systems such as spectrophotometric or conductometric detectors. MicroTAS can be also used in biochemistry for DNA chip analysis and drug discovery studies.

Microfactories provide micro-scaled production. This involves parallel production. Explosive reactions or reaction demanding intensive heat exchange can be divided into safer microreactions, but still providing the same volume of production.

Microseparation systems are miniaturized separation systems.

Point-of-care (POC) devices involve diagnostic testing carried out when a patient visits the clinic, with the results available at that visit. Such devices usually consist of a disposable test cartridge and a reading device, usually hand-held or desktop-sized.

Microfluidic devices can be fabricated from a variety of materials. Silicon (Si) has been used extensively in microfluidic devices. Silicon can be an especially good material for microfluidic channels coupled with microelectronics or other microelectromechanical systems (MEMS). It also has good stiffness, allowing the formation of fairly rigid microstructures, which can be useful for dimensional stability. In these applications, the silicon surface is actually a silicon oxide that naturally forms upon exposure of silicon to air or that is formed by another oxidation method. When a material is referred to as “silicon,” the material can include silicon bearing such an oxide surface.

Generally, a photoresist is spun onto a silicon substrate. The photoresist is then exposed to ultraviolet (UV) light through a high-resolution mask with the desired device patterns. After removing the excess unpolymerized photoresist, the silicon wafer is placed in a wet chemical etching bath that anisotropically etches the silicon in locations not protected by photoresist, resulting in a silicon wafer in which microchannels are etched. A glass coverslip can be used to fully enclose the channels and holes are drilled in the glass to allow fluidic access. For straighter edges and a deeper etch depth, deep reactive ion etching (DRIE) is an alternative to wet chemical etching.

Another material suitable for microfluidic device is polydimethylsiloxane (PDMS). Generally, liquid PDMS is poured over a mold and cured to cross-link the polymer, resulting in an optically clear, relatively flexible material that can be stacked onto other cured polymer slabs to form complex three dimensional geometries.

1. Surface Wetting

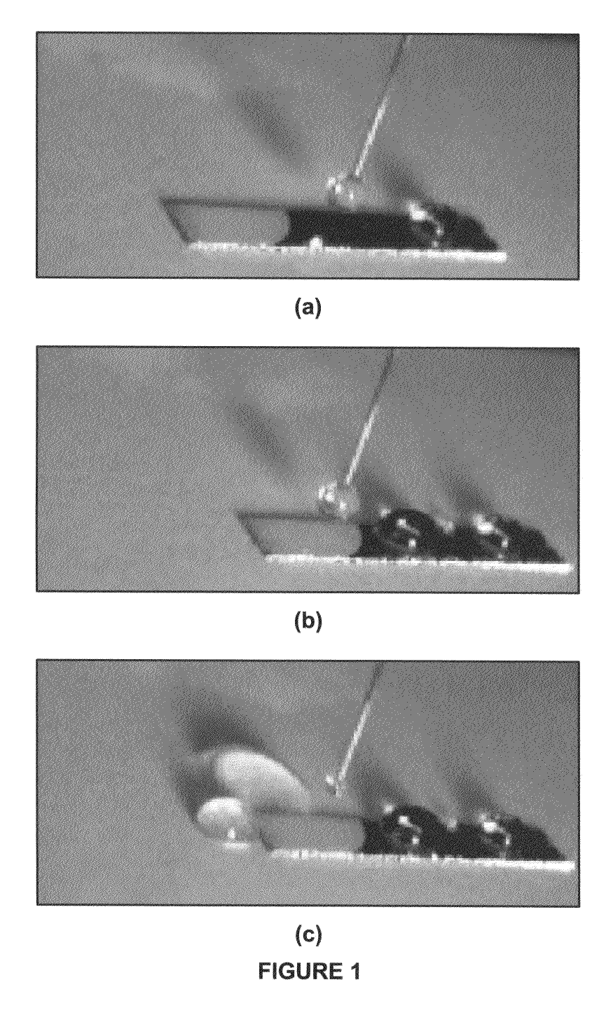

Over the past 70 years, pioneers such as Wenzel and Cassie and Baxter have made notable contributions to the understanding of surface wetting. For further discussion, see Cassie, A. and S. Baxter, Wettability of porous surfaces. Trans. Faraday Soc., 1944. 40: p. 546-551; Wenzel, R. N., Resistance of solid surfaces to wetting by water. Industrial and Engineering Chemistry, 1936. 28: p. 988-994, both of which are herein incorporated by reference. Recently there has been a renewed interest in this subject and researchers have concentrated their attention on nanostructured materials, actuation of liquid contact angle changes using external fields, and surface analysis measurements. A major goal in this area has been to control phenomena related to wetting such as capillary rise and fall and the movement of liquids along surfaces using an external stimulus such as light or electric fields. The interest in studies focused on water resides on the obvious ubiquity of the fluid and its importance in biomedicine and environmental studies. It was noted that even though plants can repel water using the so-called Lotus effect, the intrinsic contact angle of their leaves can be below 90 degrees. This effect was indicated to be non-ergodic since the same leaf can be fully wetted or non-wetted depending upon its history. An explanation for this phenomenon is that leaf surfaces feature roughness at multiple length scales. For further discussion, see Otten, A. and S. Herminghaus, How Plants Keep Dry: A Physicist's Point of View. Langmuir, 2004. 20(6): p. 2405-2408, which is herein incorporated by reference. This property can be mimicked to create artificial superhydrophobic surfaces. For further discussion, see Lai, S. C. S., University of Leiden, Mimicking nature: Physical basis and artificial synthesis of the Lotus-effect 1-31 (2003), http://home.wanadoo.n1/scslai/lotus.pdf, which is herein incorporated as Appendix C. When placed over a superhydrophobic surface, water drops tend to minimize their contact with the surface by becoming spherical, and tend to slide or roll off the surface extremely easily, as if they were repelled by the surface. The Lotus effect can be described by the photographs in FIG. 1.

2. Roughness

One approach to preparing microscopically rough surfaces has been the use of photolithographic methods. For example, standard photolithography with a resist can be used to prepare surfaces with defined surface feature (pillar arrays) dimensions in an n-type silicon substrate. The height of the surface features, h, is specified by the etch depth.

In another approach, x-ray lithography techniques, such as (S)LIGA, can be used to define high aspect ratio structures in nickel. The process consists of exposing a sheet of PMMA bonded to a wafer using X-ray lithography. The PMMA is then developed and the exposed material is removed. Nickel is then electroplated up in the open areas of the PMMA. The nickel over-plate is removed by polishing, leaving high aspect ratio nickel parts. The PMMA is removed, and the nickel parts may remain anchored to the substrate or be released.

Rough surfaces including surface features can be prepared by physical vapor deposition methods that include, for example, evaporation and sputtering.

In evaporative methods, a substrate can be placed in a high vacuum chamber at room temperature with a crucible containing the material to be deposited. A heating source can be used to heat the crucible causing the material to evaporate and condense on all exposed cool surfaces of the vacuum chamber and substrate. Typical sources of heating include, for example, e-beam, resistive heating, RF-inductive heating. The process typically can be performed on one side of the substrate at a time. In some systems, the substrate can be heated during deposition to alter the composition/stress of the deposited material.

In sputtering methods, a substrate can be placed in a vacuum chamber with a target (a cathode) of the material to be deposited. A plasma is generated in a passive source gas (e.g., Argon) in the chamber, and the ion bombardment is directed towards the target, causing material to be sputtered off the target and condense on the chamber walls and the substrate. A strong magnetic field can be used to concentrate the plasma near the target to increase the deposition rate. The ejection of atoms or groups of atoms from the surface of the cathode of a vacuum tube can be the result of heavy-ion impact. Sputtering methods can be used to deposit a thin layer of metal on a glass, plastic, metal, or other surface in a vacuum.

Chemical vapor deposition (CVD) methods can also be used to prepare rough surfaces. CVD methods pertain to the growth of thin solid films on a crystalline substrate as the result of thermo chemical vapor-phase reactions. CVD methods include, for example, low-pressure chemical vapor deposition (LPCVD) and plasma enhanced chemical vapor deposition (PECVD).

LPCVD can be performed in a reactor at temperatures up to about 900° C. A deposited film is a product of a chemical reaction between the source gases supplied to the reactor. The process typically can be performed on both sides of the substrate at the same time.

PECVD can be performed in a reactor at temperatures up to about 400° C. The deposited film is a product of a chemical reaction between the source gases supplied to the reactor. A plasma is generated in the reactor to increase the energy available for the chemical reaction at a given temperature. The process typically can be performed on one side of the substrate at a time.

In the present methods, multidimensional rough surfaces can be prepared as disclosed herein.

Surface energy gradients can be designed by preparing surfaces having varying degrees of roughness. For example, chemically homogeneous surfaces of varying roughness can be prepared by photolithographic techniques. To prepare a surface roughness gradient, for example, substantially parallel strips of surfaces can be prepared and positioned so that fluid droplets in contact with the surface will contact at least two strips along the surface roughness gradient. Surface features are typically at least one order of magnitude smaller than the fluid droplet size. The strips can be selected such that each strip has a successively greater surface roughness. A path that is substantially perpendicular to the strips, therefore, constitutes a gradient of surface roughness. In such a system, the fluid droplet sequentially contacts strips of increasing roughness as it moves from strips of lower roughness to strips of greater roughness, thereby successively minimizing its contact angle with the surface as roughness increases.

3. Surface Tension Driven Microfluidic Systems

At the microscale, surface tension becomes a relatively large force, as compared to other forces such as gravity or structural stiffness. In mechanical devices, surface tension begins to dominate other forces when physical features are shrunk to micrometers. Electrocapillary and electrowetting actively use surface tension at the microscale. Electrowetting is an electrically-induced change of a material's wettability.

Surface tension driven microfluidic systems employ surface tension to generate motion in fluid droplets. For example, hydrophobic and hydrophilic interactions of the fluid droplet with the system surface drive the droplet from regions of greater hydrophobicity (lower hydrophilicity) to regions of lower hydrophobicity (greater hydrophilicity) along a gradient of successively decreasing hydrophobicity (increasing hydrophilicity).

Tortuous Solid-Liquid-Gas Contact Line

Fractally rough surfaces generally provide a highly involved and intricate interface with fluid droplets in contact with the surface. The contact angle at the interface between the fractally rough surface with hydrophobic surface coating and the fluid droplet can be high, often approaching the theoretical maximum of a 180° apparent contact angle. Accordingly, fractally rough surfaces possess a smaller level of contact angle hysteresis when superhydrophobic than well-ordered surfaces or surfaces that are rough at the microscale but not at the nanoscale.

5. Ratio of Surface Area to Volume

Generally, at the microscale, for example in microfluidic devices, the ratio of surface area to volume of a given liquid is extremely high compared to the ratio of surface area to volume at normal scales. Accordingly, surface properties and interactions begin to dominate other properties and interactions.

6. Fluid Droplet

The liquids as disclosed herein can be in the form of drops or droplets which represent discreet self contained units of the liquid. The drops and droplets can be any size, such as the sizes disclosed herein. The word “drop” or “droplet,” when applied to a fluid, can include any discrete portion of fluid, including a free standing drop or portion on a surface, a portion of fluid in a capillary, channel, or similar partially confined space, and fluid portions within a porous medium.

7. Contact Angle

The contact angle between a fluid droplet and a surface generally refers to the pure water equilibrium contact angle. Advancing angles generally follow Cassie-Baxter wetting with a constant fraction of the surface wetted for a particular roughness, while receding angles generally follow Wenzel wetting. Due to contact with the surface, Wenzel wetting creates the condition for water drop movement. The photowetting driving force is proportional to roughness.

8. Contact Angle Hysteresis

The contact angle hysteresis is the difference between the advancing contact angle and the receding contact angle in resistance to motion of the fluid droplet. If the contact angle hysteresis is larger than the induced contact angle change, contact angle hysteresis occurs, and movement of the fluid is slowed or stopped.

This hysteresis effect can be caused by the interaction of the receding edge with the surface. For example, attractive interactions between the surface and the fluid at the receding edge can retard motion of the fluid droplet. Hysteresis can make the driving force smaller and hence slow the speed of movement. Hysteresis can be overcome by using very rough surfaces in combination with surface modification by hydrophobic molecules. At a constant velocity the driving force equals the drag force; hence, the smaller the drag force the lower the velocity, a small difference results in a slower velocity.

9. reduced contact angle hysteresis

Superhydrophobic rough surfaces provide a reduced contact angle hysteresis when used in surface-tension driven microfluidic applications. For further discussion, see Lafitma, A. & Quere, D., Superhydrophobic states. Nature Materials 2, 457-460 (2003); Bico, J., Marzolin, C. & Quere, D. Pearl drops. Europhysics Letters 47, 220-226 (1999); and Shin, J.-Y., Kuo, C.-W., Chert, P. & Moth C.-Y. Fabrication of tunable superhydrophobic surfaces by nanosphere lithography, Chemistry of Materials 16, 561-564 (2004) all of which are herein incorporated by reference. For additional discussion of fabrication of tunable superhydrophobic surfaces by nanosphere lithography, see Chemistry of Materials 16, 561-564 (2004), which is herein incorporated by reference. One reason for the small degree of hysteresis is the very low solid-surface free energy resulting from the hydrophobic molecular coating. For additional discussion, see Chibowski, E., Surface free energy of a solid from contact angle hysteresis. Advances in Colloid and Interface Science 103, 149-172 (2003), which is herein incorporated by reference.

Fractally-rough surfaces are particularly interesting for microfluidic applications as there are indications that these surfaces possess a smaller level of contact angle hysteresis than well-ordered ones. For further discussion, see Shin, J.-Y., Kuo, C.-W., Chert, P. & Moth C.-Y. Fabrication of tunable superhydrophobic surfaces by nanosphere lithography, Chemistry of Materials 16, 561-564 (2004); Ramos, S. M. M., Charlaix, E. & Benyagoub, A., Contact angle hysteresis on nano-structured surfaces, Surface Science 540, 355-362 (2003), both of which are herein incorporated by reference. This phenomenon can be due to the instability of the three-dimensional, tortuous solid-liquid-gas contact line in randomly rough surfaces as compared to that in well-ordered two-dimensional rough surfaces.

Accordingly, photoresponsive monolayer coatings on fractally rough, superhydrophobic surfaces can exhibit contact angle magnification and lowered contact angle hysteresis. Using this approach, contact angle amplification and hysteresis reduction were improved by as much as a factor of two.

In an alternative aspect, the fluid droplet can comprise a liquid other than water. For example, the fluid droplet can be a nonpolar liquid such as an oil or an organic solvent. In this aspect, the fractally rough silicon nanowire-bearing surfaces can be used as suitably rough surfaces. Likewise, the disclosed spiropyrans can be used as a photosensitive variable hydrophobicity agent in this aspect.

However, in order to minimize solid-surface free energy and interactions with the nonpolar droplet, and therefore minimize contact angle hysteresis for the nonpolar fluid droplet, a hydrophilic (polar) surface coating can be used.

Exemplary hydrophilic coating materials can include ethylene glycol, ethylene glycol derivatives, polyethylene glycol, polyethylene glycol derivatives, polyvinylpyrrolidone, polyvinylpyrrolidinone derivatives, and the like.

Hydrophilic surfaces can also be prepared by contacting silicon surfaces with diluted sulfuric acid, nitric acid, or hydrofluoric acid, thereby producing a top layer consisting of hydroxyl moieties on the oxide surface. In this aspect, a nonpolar fluid droplet placed upon a suitably rough surface that has been coated with a substance that repels the nonpolar solvent can be induced to move provided the advancing contact angle is less than the receding contact angle.

### C. DEVICES

In one aspect, the invention relates to a digital magnetofluidic device including a superhydrophobic surface; a magnetically active fluid droplet in contact with the surface; and a magnetic field coupled with at least a portion of the droplet. It is understood that the devices can be used in combination with the methods.

1. Surfaces

In one aspect, the superhydrophobic surface comprises at least two regions of differing hydrophobicity. In a further aspect, the superhydrophobic surface comprises a wettability gradient. For further discussion, see Lu et al., “Low-density polyethylene (LDPE) surface with a wettability gradient by tuning its microstructures,” Macromolecular Rapid Communications, 2005, 26 (8), 637-642, which is herein incorporated by reference. In a further aspect, the superhydrophobic surface comprises at least two different superhydrophobic materials having differing superhydrophobicities. In a yet further aspect, the superhydrophobic surface comprises at least two superhydrophobic materials having differing roughnesses.

Generally, the superhydrophobic surface can be any superhydrophobic surface known by those of skill in the art and can be prepared by any method known to those of skill in the art. As an example, the superhydrophobic surface can comprise poly(tert-butyl acrylate)-block-poly(dimethylsiloxane)-block-poly(tert-butyl acrylate) (PtBA-b-PDMS-b-PtBA). For further discussion, see Han et al., “Diverse Access to Artificial Superhydrophobic Surfaces Using Block Copolymers,” Langmuir, 2005, 21, 6662-6665, which is herein incorporated by reference.

As a further example, the superhydrophobic surface can comprise superhydrophobic isotactic polypropylene. For further discussion, see Erbil et al., “Transformation of a Simple Plastic into a Superhydrophobic Surface,” Science, 2003, 299, 1377-1380, which is herein incorporated by reference.

As a further example, the superhydrophobic surface can comprise superhydrophobic boehmite (AlOOH) or superhydrophobic silica (SiO₂). Such surfaces can be prepared by sublimation of aluminum acetylacetonate according to the procedure of Nakajima et al., “Transparent Superhydrophobic Thin Films with Self-Cleaning Properties,” Langmuir, 2000, 16, 7044-7047, which is herein incorporated by reference.

As a further example, the superhydrophobic surface can comprise a superhydrophobic fluorine-containing nanocomposite coating prepared from a sol gel prepared from tetraethoxysilane, 1H,1H,2H,2H-perfluorooctyltriethoxysilane, and silica. For further discussion, see Pilotek et al., “Wettability of Microstructured Hydrophobic Sol-Gel Coatings,” Journal of Sol-Gel Science and Technology, 2003, 26, 789-792, which is herein incorporated by reference.

As a further example, the superhydrophobic surface can comprise polytetrafluoroethylene (PTFE) coated mesh film. For further discussion, see Feng et al., “A Superhydrophobic and Super-Oleophilic Coating Mesh Film for the Separation of Oil and Water,” Angew. Chem. Int. Ed., 2004, 43, 2012-2014, which is herein incorporated by reference.

As a further example, the superhydrophobic surface can comprise fluorinated dislocation-etched aluminum. For further discussion, see Qian, B. et al., “Fabrication of Superhydrophobic Surfaces by Dislocation-Selective Chemical Etching on Aluminum, Copper, and Zinc Substrates,” Langmuir, 2005, 21, 9007-9009, which is herein incorporated by reference.

As a further example, the superhydrophobic surface can comprise a multiplicity of carbon nanotubes. For further discussion, see Lau et al., “Superhydrophobic Carbon Nanotube Forests,” Nano Letters, 2003, 3(12), 1701-1705, which is herein incorporated by reference.

As a further example, the superhydrophobic surface can comprise a multiplicity of carbon nanotubes coated with polytetrafluoroethylene (PTFE). For further discussion, see Lau et al., “Superhydrophobic Carbon Nanotube Forests,” Nano Letters, 2003, 3(12), 1701-1705, which is herein incorporated by reference.

As a further example, the superhydrophobic surface can comprise a multiplicity of carbon nanotubes coated with a zinc oxide thin film. For further discussion, see Huang et al., “Stable Superhydrophobic Surface via Carbon Nanotubes Coated with a ZnO Thin Film,” J. Phys. Chem., 2005, 109, 7746-7748, which is herein incorporated by reference.

As a further example, the superhydrophobic surface can comprise a multiplicity of superhydrophobic amphiphilic poly(vinyl alcohol) (PVA) nanofibers. Such surfaces can be prepared using the template-based extrusion method of Feng et al., “Creation of a Superhydrophobic Surface from an Amphiphilic Polymer,” Angew. Chem. Int. Ed., 2003, 42, 800-802, which is herein incorporated by reference.

As a further example, the superhydrophobic surface can comprise anode oxidized aluminum. For further discussion, see Shibuichi et al., “Super Water- and Oil-Repellant Surfaces resulting from Fractal Structure,” Journal of Colloid and Interface Science, 1998, 208, 287-294, which is herein incorporated by reference.

As a further example, the superhydrophobic surface further can comprise a superhydrophobic coating including residues of 1H,1H,2H,2H-perfluorooctyltrichlorosilane or 1H,1H,2H,2H-perfluorodecyltrichlorosilane. For further discussion, see Shibuichi et al., “Super Water- and Oil-Repellant Surfaces resulting from Fractal Structure,” Journal of Colloid and Interface Science, 1998, 208, 287-294, which is herein incorporated by reference.

As a further example, the superhydrophobic surface can comprise a superhydrophobic micropatterned polymer film having micro- or nano-scale surface concavities. For further discussion, see Wang et al., “Phase-Separation-Induced Micropatterned Polymer Surfaces and Their Applications,” Adv. Funct. Mater., 2005, 15, 655-663, which is herein incorporated by reference.

As a further example, the superhydrophobic surface can comprise a superhydrophobic porous poly(vinylidene fluoride) membrane. For further discussion, see Peng et al., “Porous Poly(Vinylidene Fluoride) Membrane with Highly Hydrophobic Surface,” Journal of Applied Polymer Science, 2005, 98, 1358-1363, which is herein incorporated by reference.

As a further example, the superhydrophobic surface can comprise superhydrophobic microstructured zinc oxide. For further discussion, see Wu et al., “Fabrication of Superhydrophobic Surfaces from Microstructured ZnO-Based Surfaces via a Wet-Chemical Route,” Langmuir, 2005, 21, 2665-2667, which is herein incorporated by reference.

As a further example, the superhydrophobic surface can comprise conductive superhydrophobic microstructured zinc oxide. For further discussion, see Li et al., “Electrochemical Deposition of Conductive Superhydrophobic Zinc Oxide Thin Films,” J. Phys. Chem. B, 2003, 107, 9954-9957, which is herein incorporated by reference.

As a further example, the superhydrophobic surface can comprise a superhydrophobic block copolymer of polypropylene and poly(methyl methacrylate). For further discussion, see Xie et al., “Facile Creation of a Bionic Superhydrophobic Block Copolymer Surface,” Adv. Mater., 2004, 16, 1830-1833, which is herein incorporated by reference.

As a further example, the superhydrophobic surface can comprise a superhydrophobic block copolymer of fluorine-end-capped polyurethane (FPU) and poly(methyl methacrylate) (PMMA). For further discussion, see Xie et al., “Facile Creation of a Super-Amphiphobic Coating Surface with Bionic Microstructure,” Advanced Materials, 2004, 16 (4), 302-305, which is herein incorporated by reference.

As a further example, the superhydrophobic surface can comprise superhydrophobic low-density polyethylene (LDPE). For further discussion, see Lu et al., “Low-Density Polyethylene (LDPE) Surface With A Wettability Gradient By Tuning Its Microstructures,” Macromolecular Rapid Communications, 2005, 26 (8), 637-642; Lu et al., “Low-density polyethylene (LDPE) surface with a wettability gradient by tuning its microstructures,” Macromolecular Rapid Communications, 2005, 26 (8), 637-642, both of which are herein incorporated by reference.

As a further example, the superhydrophobic surface can comprise a superhydrophobic film deposited by microwave plasma-enhanced chemical vapor deposition (MPECVD) of trimethyltrimethoxysilane (TMMOS) and carbon dioxide. For further discussion, see Wu et al., “Mechanical Durability Of Ultra-Water-Repellent Thin Film By Microwave Plasma-Enhanced CVD,” Thin Solid Films, 2004, 457 (1), 122-127, which is herein incorporated by reference.

As a further example, the superhydrophobic surface can comprise a superhydrophobic polystyrene microsphere/nanofiber composite film (PMNCF). For further discussion, see Jiang et al., “A lotus-leaf-like superhydrophobic surface: A porous microsphere/nanofiber composite film prepared by electrohydrodynamics,” Angew. Chem. Int. Ed., 2004, 43(33), 4338-4341, which is herein incorporated by reference.

As a further example, the superhydrophobic surface can comprise a superhydrophobic coating including residues of 2-(3-(triethoxysilyl)propylaminocarbonylamino)-6-methyl-4[1H]pyrimidinone. For further discussion, see Han et al., “Fabrication of Superhydrophobic Surface from a Supramolecular Organosilane with Quadruple Hydrogen Bonding,” J. Am. Chem. Soc., 2004, 126, 4796-4797, which is herein incorporated by reference.

As a further example, the superhydrophobic surface can comprise a superhydrophobic calcium carbonate and poly(N-isopropyl acrylamide) hierarchical structure. For further discussion, see Zhang et al., “Fabrication of Superhydrophobic Surfaces from Binary Colloidal Assembly,” Langmuir, 2005, 21, 9143-9148, which is herein incorporated by reference.

As a further example, the superhydrophobic surface can comprise superhydrophobic electrospun polystyrene trichomelike structures. For further discussion, see Gu et al., “Artificial silver ragwort surface,” Applied Physics Letters, 2005, 86, 201915, which is herein incorporated by reference.

As a further example, the superhydrophobic surface can comprise a superhydrophobic copolymer including poly((3-trimethoxysilyl)propyl methacrylate-r-polyethylene glycol methyl ether methacrylate) (poly(TMSMA-r-PEGMA)). For further discussion, see Suh et al., “Control Over Wettability of Polyethylene Glycol Surfaces Using Capillary Lithography,” Langmuir, 2005, 21, 6836-6841, which is herein incorporated by reference.

As a further example, the superhydrophobic surface can comprise microscale features produced by sol-gel etching. For further discussion, see Smoukov et al., “Cutting into Solids with Micropatterned Gels,” Advanced Materials, 2005, 17, 1361-1365, which is herein incorporated by reference.

Superhydrophobic surfaces that combine superhydrophobic molecular coatings with surface roughness are generally characterized by either well-ordered microstructures, as described in Lafitma, A. & Quere, D., Superhydrophobic states. Nature Materials 2, 457-460 (2003); Bico, J., Marzolin, C. & Quere, D. Pearl drops. Europhysics Letters 47, 220-226 (1999), both of which are herein incorporated by reference, or by random fractal geometry, as described in Onda, T., Shibuichi, S., Satoh, N. & Tsujii, K., Super-water-repellent fractal surfaces, Langmuir 12, 2125-2127 (1996); Shibuichi, S., Yamamoto, T., Onda, T. & Tsujii, K., Super water- and oil-repellent surfaces resulting from fractal structure, Journal of Colloid and Interface Science 208, 287-294 (1998), both of which are herein incorporated by reference. Rough fractal surfaces are particularly interesting due to the extremely high degree of roughness that they possess.

The liquid contact angle on a solid surface is related to the interfacial energy and roughness. The dependence of the apparent solid-liquid contact angle on surface roughness in terms of flat-surface contact angle can be described by the Cassie model, as described in Cassie, A. B. & Baxter, S., Wettability of porous surfaces, Transactions of the Faraday Society 40, 546-551 (1944), which is herein incorporated by reference, and the Wenzel model, as described in Wenzel, R. N., Resistance of solid surfaces to wetting by water, Industrial and Engineering Chemistry Research 28, 988-994 (1936), which is herein incorporated by reference.

Wenzel, in Wenzel, R. N., Resistance of solid surfaces to wetting by water, Industrial and Engineering Chemistry Research, 1936, 28: p. 988-994, which is herein incorporated by reference, and Cassie, in Cassie, A. B. and Baxter, S., Wettability of porous surfaces, Transactions of the Faraday Society 1944. 40: p. 546-551, which is herein incorporated by reference, developed approaches to model the rough surface contact angle, θᵣ, using average roughness characteristics of the surface. Wenzel approached the problem by assuming that the liquid filled every part of the rough surface in the region of its contact. Cassie, on the other hand, assumed that the features on the surface would lift up the liquid in the region of contact, leading to the formation of a composite surface.

Both Cassie and Wenzel types of wetting represent local energy minima for drops on rough surfaces. For further discussion, see Patankar, N. A., On the modeling of hydrophobic contact angles on rough surfaces, Langmuir 19, 1249-1253 (2003), which is herein incorporated by reference. Drops gently deposited onto superhydrophobic rough surfaces resulted in extremely high contact angles which were well represented by the Cassie model, whereas drops that were allowed to fall onto the surface from a height gave lower contact angles that were better represented by the Wenzel model. For further discussion, see He, B., Patankar, N. A. & Lee, J., Multiple equilibrium droplet shapes and design criterion for rough hydrophobic surfaces, Langmuir, 19, 4999-5003 (2003), which is herein incorporated by reference.

Contact angles on rough surfaces can transition from Cassie to Wenzel behavior when pressure is applied to the drop. For further discussion, see Bico, J., Marzolin, C. & Quere, D., Pearl drops. Europhysics Letters 47, 220-226 (1999), which is herein incorporated by reference. It was found that when using smaller drops (˜5 μL), visible irradiation of the coated nanowire surface resulted in advancing contact angles of greater than 170°. Larger drops (˜15 μL) produced advancing contact angles of 157°. The weight of the larger drops can force the liquid into the depressions in the surface, making the Wenzel model applicable under these conditions.

a. Cassie Model

Cassie's model is based on the assumption that the liquid does not fill the crevices of the rough surface, but rests on a composite surface composed of the solid material and air.

b. Wenzel Model

In contrast, Wenzel's model hypothesizes that the liquid completely fills the depressions in the rough surface over the projected area of solid-liquid contact.

In the Wenzel model, the apparent contact angle on the fractal surface, θ_{f} may be expressed as,

$$cos {θ}_{f} = {( \frac{L}{l} )}^{D - 2} cos θ \qquad ( Eqn . 1 )$$

where θ is the contact angle on a flat surface with identical chemistry and D is the fractal dimension of the surface between the upper and lower scale limits, L and 1, respectively. This indicates that if the flat surface contact angle is changed from a value θ₁ to θ₂ by the action of an external stimulus such as light, the apparent contact angle change on the fractal surface (θ_{f1}−θ_{f2}) may be expressed by

$$( cos {θ}_{f 1} - cos {θ}_{f 2} ) = {( \frac{L}{l} )}^{D / 2} ( cos {θ}_{1} - cos {θ}_{2} ) \qquad ( Eqn . 2 )$$

Since the term (L1l)^{D-2} is always>1 for a rough surface, this indicates that the use of a superhydrophobic rough surface will always amplify the magnitude of the stimulus-induced contact angle change relative to the smooth surface (until the theoretical limit of a 180° contact angle is reached). Thus, by combining photoswitchable surface chemistry with control of surface morphology, it is possible to amplify the photo-induced changes in the water contact angle.

Each of these theories leads to different values of θᵣ, and it has been experimentally demonstrated that both the Cassie angle, θᵣ^{C}, and the Wenzel angle, θᵣ^{W}, can be formed on the same surface depending on the method use to form the droplet. For further discussion, see He, B., Patankar, N. A., and Lee, J., Multiple equilibrium droplet shapes and design criterion for rough hydrophobic surfaces, Langmuir, 2003. 19: p. 4999-5003, which is herein incorporated by reference. It has also been experimentally confirmed in different systems, that the Cassie and Wenzel contact angles each represent local energy minima separated by energy barriers corresponding to partial wetting of the roughness features. For further discussion, see id. and Bico, J., Marzolin, C. and Quéré, D., Pearl drops, Europhysics Letters, 1999. 47(2): p. 220-226, which is herein incorporated by reference.

The global energy minimum for this system was calculated to be the smaller of the two contact angles, θᵣ^{C} and θᵣ^{W}. Experimental measurements on PDMS microstructures showed some quantitative agreement with this calculation. Differences between the experimental and calculated values were attributed to the 30° contact angle hysteresis present on even “flat” PDMS surfaces.

The interfacial energies of spiropyran-coated surfaces can be changed solely by altering the wavelength of irradiation. For further discussion, see Rosario, R. et al., Photon-modulated wettability changes on spiropyran-coated surfaces, Langmuir 18, 8062-8069 (2002). The maximum difference obtained between water contact angles under UV and Visible light was of the order of 13°.

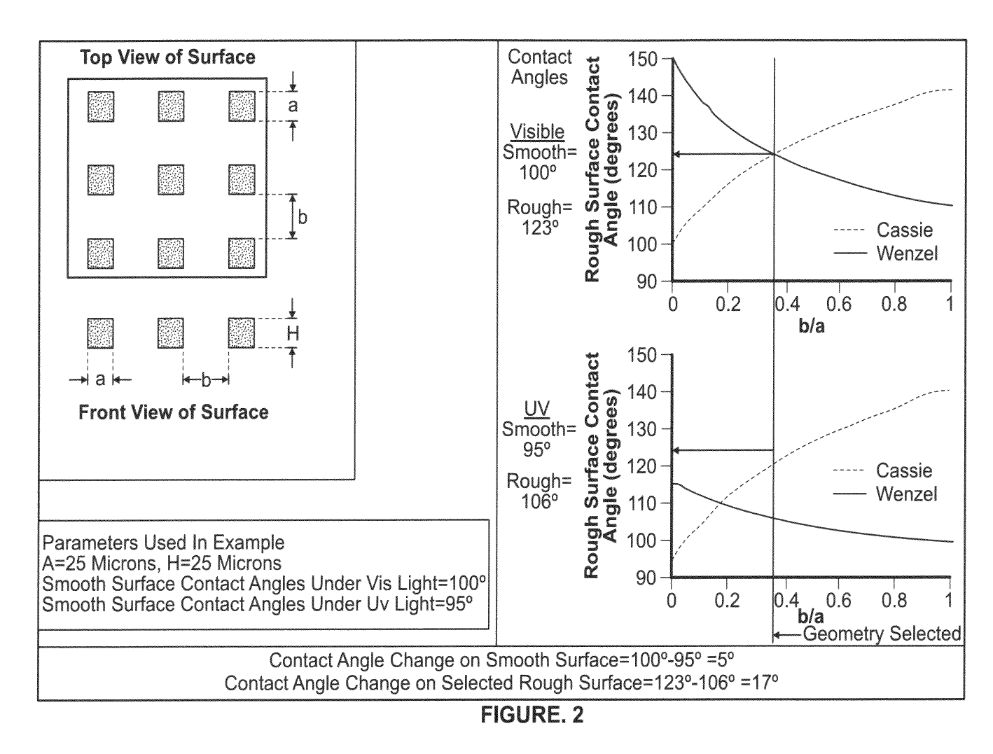

Selection of a particular surface roughness allows amplification of the magnitude of the light-induced contact angle changes on spiropyran-coated surfaces. The roughness characteristics are defined by the geometry of surface features. FIG. 2 shows an example of how the selection of a particular rough surface can increase the light-induced contact angle change. In this example a 5° light-induced change is predicted to be amplified into a 17° change in contact angle on the rough surface.

The apparent contact angle on non-porous Euclidian rough surfaces is given by the Wenzel equations. For further discussion, see Wenzel, R. N., Industrial and Engineering Chemistry Research, 1936, 28, 988, which is herein incorporated by reference.

cos θ_{W}=r cos θs (Eqn. 3)

The roughness coefficient r is defined as the ratio of the actual solid-liquid interfacial area to the projected solid-liquid interfacial area, and θ_{W} and θ_{S} are the solid-liquid contact angles on the rough surface and smooth surface, respectively. The effect of r is to enhance the inherent wetting behavior of the surface (by increasing the contact angle>90°, and decreasing the contact angle<90°).

However, for fractal surfaces, the term r is very large and can even be infinite for a mathematically ideal fractal surface. Additionally, if the fractal behavior extends to the molecular scale, fluids having different molecular dimensions would experience different solid-liquid interfacial areas. Thermodynamic models for the equilibrium contact angle, which take into account both the fractal nature of the surface and the relative dimensions of the different fluid molecules, have been developed. For further discussion, see Hazlett, R. D., Journal of Colloid and Interface Science, 1990, 137, 527, which is herein incorporated by reference. The equilibrium contact angle is given by

$$cos {θ}_{fractal} = [ ( \frac{1 - Γ {f}^{1 - D / 2}}{1 - Γ} ) {( \frac{{σ}_{1}}{{σ}_{R}} )}^{1 - D / 2} ] cos {θ}_{S} \qquad ( Eqn . 4 )$$

Here, f≡(σ₂/σ₁) and Γ≡(γₛ₂/γₛ₁). σ refers to the area of the interfacial tension, D is the fractal dimension, and the subscripts s, l, and 2 refer to the surface, liquid, and vapor, respectively. σ_{R} is a reference area that represents the scale that would yield the Euclidean area if the fractal nature and dimension held to this scale, such that

σR² sin² θ=C σ_{R}^{1-D/2} (Eqn. 5)

where R is the radius of the drop.

The first term within the correction factor in Eqn. 5 can either depress or elevate the contact angle depending on the relative sizes of the fluid molecules and their wetting tendencies. The second term is a measure of the extent of the fractal nature of the surface and is always greater than 1. When the lower limit of fractal behavior is larger than the areas of the fluid molecules, then the fluid molecules are able to probe all the irregularities on the surface and Eqn. 4 reduces to

$$cos {θ}_{W , fractal} = {( \frac{L}{l} )}^{D / 2} cos {θ}_{S} \qquad ( Eqn . 6 )$$

where L and 1 are the upper and lower limits of fractal behavior. The correction term here is analogous to the roughness correction term of the Wenzel equation and quantifies the ratio of the actual solid surface area to the projected surface area. For example, and alkyl ketene dimmer fractal surface was found to possess a correction factor of (L/l)^{D-2}=(34/0.2)^{2.29-2}≅4.43, with the fractal limits being expressed in microns. For further discussion, see Onda, T., Shibuichi, S., Satoh, N. & Tsujii, K., Super-water-repellent fractal surfaces, Langmuir 12, 2125-2127 (1996), which is herein incorporated by reference.

In the case of rough porous surfaces, Cassie's equation describes the equilibrium contact angle on a composite surface, which retains pockets of air underneath the sessile drop. For further discussion, see Cassie, A. B. & Baxter, S., Wettability of porous surfaces, Transactions of the Faraday Society 40, 546-551 (1944), which is herein incorporated by reference.

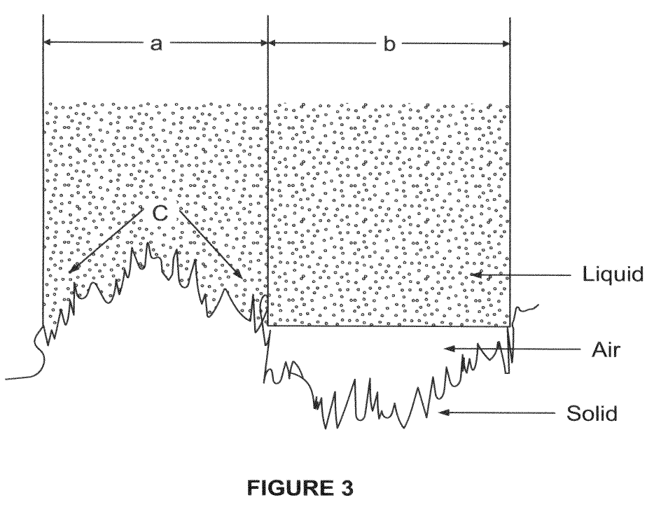

cos θ_{C}=f cos θ₁+f₂ (Eqn. 7)

where f₁ and f₂ are the ratios of projected areas of the solid surface-liquid and air surface-liquid interfaces, respectively, to the total projected area, θ_{C} is the Cassie contact angle and θ₁ is the solid-liquid contact angle. FIG. 3 shows a drop of liquid sitting on a fractally rough composite surface made up of solid and air. From the figure, f₁=a/(a+b)=f and f₂=b/(a+b)=(1−f). Substituting these ratios into Eqn. 7, the equation becomes

cos θ_{C}=f cos θ₁+f−1 (Eqn. 8)

Since the surface is fractally rough, in this case, θ₁ is equivalent to the Wenzel contact angle on fractally rough surfaces as given by Eqn. 6. Therefore, the Cassie equation can be extended to uniformly heterogeneous fractal surfaces by substituting Eqn. 6 into Eqn. 8.

$$cos {θ}_{C , fractal} = {f ( \frac{L}{l} )}^{D - 2} cos {θ}_{S} + f - 1 \qquad ( Eqn . 9 )$$

This is the equivalent fractal form of Cassie's equation. While f can be calculated for well-defined Euclidean surfaces, fractal surfaces are not amenable to this quantitative treatment.

Both Wenzel and Cassie equations represent local energy minima in drop conformation. For fractal surfaces, the Wenzel contact angle is always lesser or equal to the Cassie contact angle. The equilibrium drop shape with the lower value of apparent contact angle on rough Euclidean surfaces will have lower energy. For further discussion, see Patankar, N. A., On the modeling of hydrophobic contact angles on rough surfaces, Langmuir 19, 1249-1253 (2003), which is herein incorporated by reference. Extending this result to fractal surfaces, the Wenzel contact angle represents the global energy minimum of the system.

At intrinsic contact angles of >90°, the apparent contact angles (Wenzel and Cassie) increase with the roughness of the surface as represented by the fractal dimension, D, until the physical limit of an apparent 180° contact angle is reached. The magnification of any light-induced contact angle change, as related to D, has a maximum at the roughness that first produces an apparent 180° contact angle on the more hydrophobic surface. Therefore, the degree of fractal surface roughness that produces the maximum magnification of light-induced contact angle changes, Dₒₚₜᵢₘₐₗ, can be predicted by

$${D}_{optimal} = \frac{ln ( - 1 / cos {θ}_{S} )}{ln ( L / l )} + 2 = \frac{ln [ sec ( {θ}_{S} + π ) ]}{ln [ L / l ]} + 2 \qquad ( Eqn . 10 )$$

d. Fractally Rough Surfaces

Cross sectional images of fractally rough oxidized silicon nanowire-bearing surfaces were obtained using SEM (FIG. 3). A box counting fractal analysis was performed on trace curves of the cross sectional SEM images, and the cross sectional fractal dimension of the surface, D_{cross}, was determined to be 1.54 between the lower and upper limits of fractal behavior of 74 nm and 202 nm, respectively. The three-dimensional fractal dimension of the surface was estimated to be D˜D_{cross}+1=2.54, as described in Vicsec, T., Fractal growth phenomena (World Scientific, Singapore, 1989), which is herein incorporated by reference.

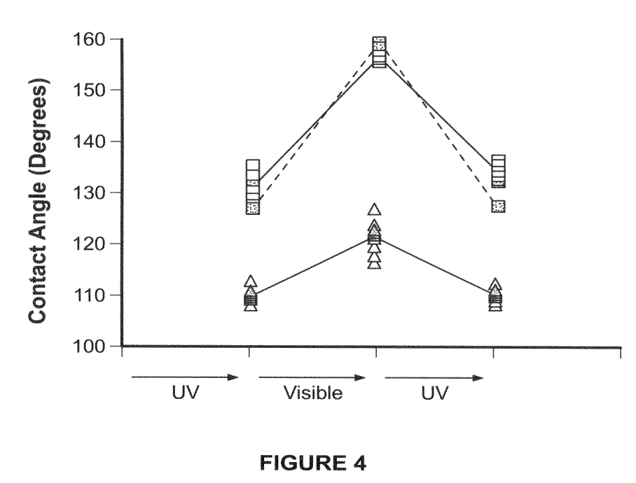

The use of these fractal roughness parameters in the Wenzel model for contact angles on fractal surfaces (Eqn. 1) gave an excellent fit with the experimental contact angles on the rough surface as shown by the dashed line in FIG. 4. This demonstrates that the large water drops filled the crevices in the nanowire structure, as described by the Wenzel model.

2. Droplets

It is understood that the fluid droplets can comprise any fluid known to those of skill in the art. It is also understood that the droplet can comprise a magnetically active fluid. In one aspect, the magnetically active fluid droplet comprises an aqueous fluid. In further aspects, the aqueous fluid comprises at least one of water, sea water, freshwater, wastewater, saliva, blood, semen, plasma, urine, lymph, serum, tears, vaginal fluid, sweat, plant or vegetable extract fluid, or cell or tissue culture media, or a mixture thereof. In yet further aspects, the magnetically active fluid droplet further comprises at least one of a biologically active agent or a pharmaceutically active agent or a mixture thereof.

A. Additives

In one aspect, the magnetically active fluid droplet further comprises ampholytes. Generally, ampholytes are chemical species of bifunctional amphoteric (both acid and basic) buffer molecules which form a pH gradient when an electric field is applied across a medium. Examples of ampholytes are glycine, lysine, ornithine, and serine; but other materials can be used.

In a further aspect, the magnetically active fluid droplet further comprises at least one of a chemically active agent, a chemical labeling agent, or a radioactive agent or a mixture thereof. Suitable chemically active agents include EDTA, carboxylic acids, amines, oxidizants, chemiluminescent reactants, and reductants A chemically active agent can be provided at a chemically effective amount. Suitable chemical labeling agents include fluorescein derivatives, rhodamine derivatives, BODIPY derivatives, eosin derivatives, and nanodots. A chemical labeling agent can be provided at an effective labeling amount. Suitable radioactive agents include radioactive isotopes of europium, iodine, phosphorous, and sulfur. A radioactive agent can be provided at a radioactively effective amount; that is, the agent can be provided in an amount sufficient for detection or sufficient to provide a desired amount of radiation.

In a further aspect, the magnetically active fluid droplet comprises at least one of a biologically active agent or a pharmaceutically active agent or a mixture thereof. Suitable pharmaceutically active agents include hormones, steroids, NO, antiviral agents, and antibiotics. A pharmaceutically active agent can be provided at a pharmaceutically effective amount. Suitable biologically active agents include biotin, DNA, RNA, antibodies, proteins, peptides, and enzymes. A biologically active agent can be provided at a biologically effective amount.

In one aspect, the magnetically active fluid droplet comprises at least one of a paramagnetic material, a diamagnetic material, or a ferromagnetic material or a mixture thereof. Suitable paramagnetic materials include particles of iron oxide, cobalt iron oxide, magnesium iron oxide, nickel, ruthenium, and cobalt. Suitable diamagnetic materials include kaolin, bentonite, barium sulfate, copper, silver, and gold particles. Suitable ferromagnetic materials include iron, iron oxide, cobalt, nickel, iron boron, and mixtures of iron oxides with copper, magnesium, and nickel oxides. In one aspect, the magnetically active fluid droplet comprises an aqueous solution or suspension of at least one of iron, nickel, or cobalt or a mixture thereof. In a further aspect, the magnetically active fluid droplet comprises an aqueous suspension of paramagnetic carbonyl iron particles.

In further aspects, at least one of a paramagnetic material, a diamagnetic material, or a ferromagnetic material or a mixture thereof is present in the droplet at a concentration of from about 0.05% (w/v) to about 5% (w/v), from about 0.1% (w/v) to about 10% (w/v), from about 0.5% (w/v) to about 5% (w/v), from about 1% (w/v) to about 10% (w/v), or from about 0.1% (w/v) to about 1% (w/v).

In a further aspect, the device can further comprise an electric field coupled with at least a portion of the droplet.

In one aspect, the particles, for example paramagnetic particles, can comprise functionalization. By functionalization, it is meant that the particles can bear chemically- or biologically-active moieties at the surface of the particle. Such moieties can be associated with the particles surface by for example covalent, noncovalent, hydrophobic, hydrophilic, hydrogen-bonding, or van der Waals interactions. For example, the functionalization can comprise at least one of a molecular recognition moiety, an optical tag, an acidic moiety, a basic moiety, a cationic moiety, and anionic moiety, a hydrophilic moiety, a hydrophobic moiety, or a stimulus-responsive molecule or a mixture thereof.

b. Contact Angle

In one aspect, the magnetically active fluid droplet has a contact angle with the superhydrophobic surface. The contact angle between the magnetically active fluid droplet and the superhydrophobic surface can be, for example, at least about 120°, at least about 130°, at least about 140°, at least about 150°, at least about 155°, at least about 160°, or at least about 165°. In various aspects, the contact angle between the magnetically active fluid droplet and the superhydrophobic surface can be from about 120° to about 180°, from about 130° to about 180°, from about 140° to about 180°, from about 150° to about 180°, from about 155° to about 180°, from about 160° to about 180°, from about 165° to about 180°, from about 140° to about 1600, from about 150° to about 170°, or about 160°. In one aspect, the contact angle is magnified relative to a smooth surface. In a further aspect, the magnetically active fluid droplet can have a contact angle hysteresis that is decreased relative to a smooth surface. In one aspect, the magnetically active fluid droplet is in motion across the surface of the superhydrophobic surface, thereby creating an advancing edge contact angle and a receding edge contact angle.

c. Under a Magnetic Field

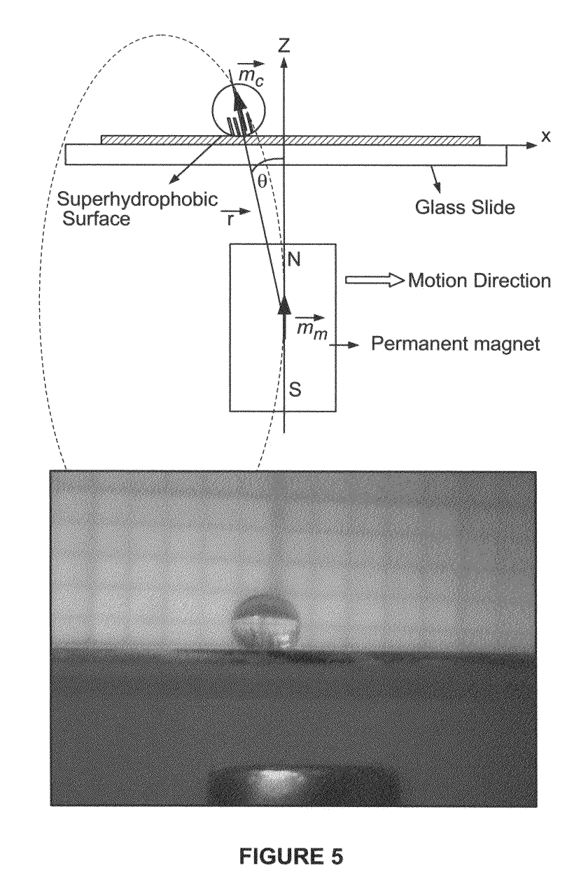

Under the influence of the magnetic field, the particles form chain-like clusters. Without wishing to be bound by theory, it is believed that the simplest way to understand this system is to consider that the permanent magnet generates a spatially non-uniform magnetic field on the region were the drop is located. This magnetic field magnetizes the paramagnetic particles that aggregate into cylindrical clusters that follow the magnetic field lines. When the magnet is displaced, the clusters move and drive the motion of the drop, as shown in FIG. 5.

It is typically very difficult to make paramagnetic-particles containing water drops perch steadily on a superhydrophobic surface. For further discussion, see Journet, C., et al., Carbon angle measurements on superhydrophobic carbon nanotube forests: effect of fluid pressure. Europhysics letters, 2005. 71(1): p. 104-109, which is herein incorporated by reference. Several methods for making drops were evaluated: spray, pipette, syringe, and capillary. Unless humidity is controlled, very small drops (1 μL sprayed drops or smaller) dry very quickly, leaving a small agglomerate of particles over the surface. Aqueous drops with paramagnetic particles with sizes in the range of 5-30 μl can be placed and stabilized on a superhydrophobic surface by the magnetic force on the paramagnetic particles exerted by a permanent magnet just below the surface. The drops were made using pipettes with plastic tips. Water drops have a higher affinity for the pipette tip than for the surface, and do not fall onto the surface even when the tip is so close to the surface that the drop bottom is in contact with the surface. If the drop contains paramagnetic particles and a magnet is placed below the surface, when the drop bottom is touching the surface the drop can be separated from the pipette tip because the drop is being held by the force exerted on the particles by the magnet. If the drop is about one millimeter away from the surface, the force on the particles may make the drop fall on the surface. If the magnetic force on the particles is strong enough, the particles are pulled out of the drop to the surface. Another technique is to place a small spot of magnetic particles on the surface (or pulled out from a different drop) can be used to make a water drop overcome its affinity for the plastic tip, thus attracting it to this point on the surface due to capillary action followed by pinning.

3. Magnetic Field

Generally, the surfaces and droplets are used in connection with a magnetic field. In one aspect, the magnetic field has a strength of at least about 0.05 nN, at least about 0.1 nN, at least about 0.2 nN, at least about 0.3 nN, at least about 0.4 nN, at least about 0.5 nN, at least about 0.6 nN, at least about 0.7 nN, at least about 0.8 nN, at least about 0.9 nN, at least about 1 nN, about 0.1 nN, about 0.2 nN, about 0.3 nN, about 0.4 nN, about 0.5 nN, about 1 nN, about 2 nN, about 5 nN, or about 10 nN.

While it is understood that the field can be produced by any method known to those of skill in the art, the magnetic field, in one aspect, is produced by a permanent magnet or an electromagnet. The field can be stationary or can be moving. In one aspect, the magnetic field is rotating.

The effect of a magnetic field on drops with varied size and particle concentrations was studied, when the magnet was placed at different positions with respect to the surface and drop. Drops with a high concentration of particles were typically deformed by the action of a magnet above or on the side of the drop. In some case, when the magnet was placed very close to the drop, the particles overcame their hold by the drop's surface tension and were pulled out before the drop moved. However, when the magnet was placed under the surface, particles inside the drop aggregated in chains and inclined towards the opposite side of the magnet, thereby following the magnetic field intensity lines. Drop movement following the magnet movement from below in linear and circular patterns was observed, for particle concentrations from 0.1% wt/wt, magnetic field intensity of approximately 0.2 kGauss to approximately 2.5 kGauss—for example from about 0.25 kG to about 1.5 kG or from about 0.5 kG to about 1.0 kG—and with speeds up to about 7 cm/sec along a 2 cm path.

### D. METHODS

Generally, the methods relate to methods for moving and controlling droplets of fluids on superhydrophobic surfaces through the use of magnetic fields. It is understood that the methods can be used in combination with the devices.

1. Linear Movement

In one aspect, the method of inducing linear movement of a fluid droplet on a surface including the acts of positioning a magnetically active fluid droplet in contact with a superhydrophobic surface; coupling a magnetic field with at least a portion of the droplet; and varying the magnetic field intensity across the surface. In a further aspect, the magnetic field has an intensity sufficient to overcome friction between the magnetically active fluid droplet and the superhydrophobic surface but insufficient to overcome the surface tension of the magnetically active fluid droplet.

In one aspect, the magnetic field has an intensity of about 0.1 in N. In a further aspect, the magnetic field has an intensity of about in N. The magnetic field can be varied, for example, so as to produce a droplet speed of about 0.5 cm/s, about 1 cm/s, about 2 cm/s, about 3 cm/s, about 4 cm/s, about 5 cm/s, about 6 cm/s, or about 7 cm/s. The maximum attainable speed remains to be determined, since the maximum speed in this experiment was limited by the maximum magnet speed. It is understood that the speed can theoretically be higher by achieving higher magnet speeds. In a further aspect, the method can further comprise the act of rotating the magnetic field, thereby subjecting the droplet to a rotational force vector.

In a further aspect, the superhydrophobic surface further can comprise at least one stimulus-responsive molecule. In such an aspect, the fluid droplet can be controlled by either magnetic stimulus or by light stimulus or both.

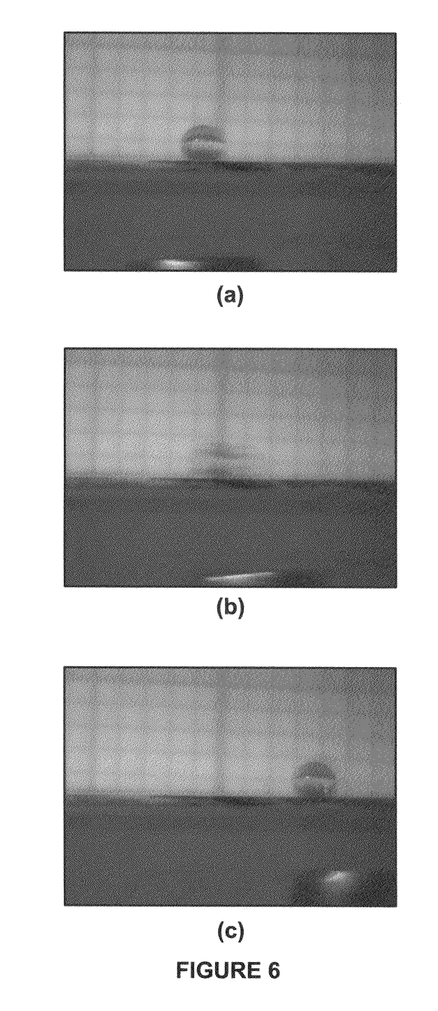

The movement of water drops on surfaces was due to the application of a magnetic field that aligns paramagnetic particles, attracts them to the magnet, and moves the drop of water in the process. Alignment of paramagnetic particles as a chain inside the droplet moves the droplet due to the rigidity of the chain. The chain distorts the shape of the drop at the bottom because the particles are attracted to the magnet and when the magnet moves the chain moves with it pushing against the “skin” or contact line of the drop due it surface tension (FIG. 6). Unlike the body force of gravity, it is believed that the imposed force can be communicated at the contact line formed by air-liquid-solid phases. The water drop movement in the system occurs in the Cassie-Baxter, mostly non-wetted mode. With the larger-sized droplets studied, without wishing to be bound by theory, it appears that the drop is sliding which likely occurs due to the lack of significant frictional resistance since the wetted contact area is low. For further discussion, see Mahadevan, L. and Y. Pomeau, Rolling droplets. Physics of fluids, 1999. 11(9): p. 2449-2453, which is herein incorporated by reference.

It was observed that the drop size does not affect the intensity of the magnetic field that is required to move drops on the superhydrophobic surface, which indicates that frictional resistance is extremely low. These results are in accordance with molecular studies that predict roughness from the nano to the micro scale at the solid-liquid interface can greatly enhance slippage, probably due to the existence of bubbles at a nano-scale at the liquid-solid interface that influence slippage. For further discussion, see Cottin-Bizonne, C. B., Jean-Louis; Bocquet, Lyderic; Charlaix, Elisabeth, Low-friction flows of liquid at nanopatterned interfaces. Nature Materials, 2003(2): p. 237-240, which is herein incorporated by reference. From measurements of the magnetic field intensity required to move the drop, it is roughly estimated that H is proportional to 1/(particle concentration)̂b, where the exponent b is in the [⅓, 2] interval.

On inclined superhydrophobic surfaces, smaller water drops actually roll down faster than larger drops which slide down the inclined surface. For further discussion, see Quéré, D. and D. Richard, Viscous drops rolling on a tilted non-wettable solid. Europhysics letters, 1999. 48(3): p. 286-291; Mahadevan, L. and Y. Pomeau, Rolling droplets. Physics of fluids, 1999. 11(9): p. 2449-2453, both of which are herein incorporated by reference. Experiments were conducted to detect whether the drops in this system “slide” or “roll.” Based on observation of hydrophobic powders placed on top of 2 mm drops, it is believed that drops of this size “slide” across the surface since the powders are not swirling even when the drops move at relatively high speeds. These drops are relatively large and they should slide when placed on an inclined superhydrophobic surface according to theoretical predictions. The drop size dynamics transition point can be interpreted as depending in part upon the wetting transition between Cassie-Baxter and Wenzel wetting modes. Cassie-Baxter wetting assumes that the drop does not penetrate the valleys caused by the roughening of the surface, while Wenzel wetting assumes that the drop does penetrate completely. However and more importantly, for smaller droplets where surface tension forces dominate over gravitational forces and when viscous effects dominate over inertia, drop rolling may occur possibly even when wetting is between the Cassie-Baxter and Wenzel regimes.

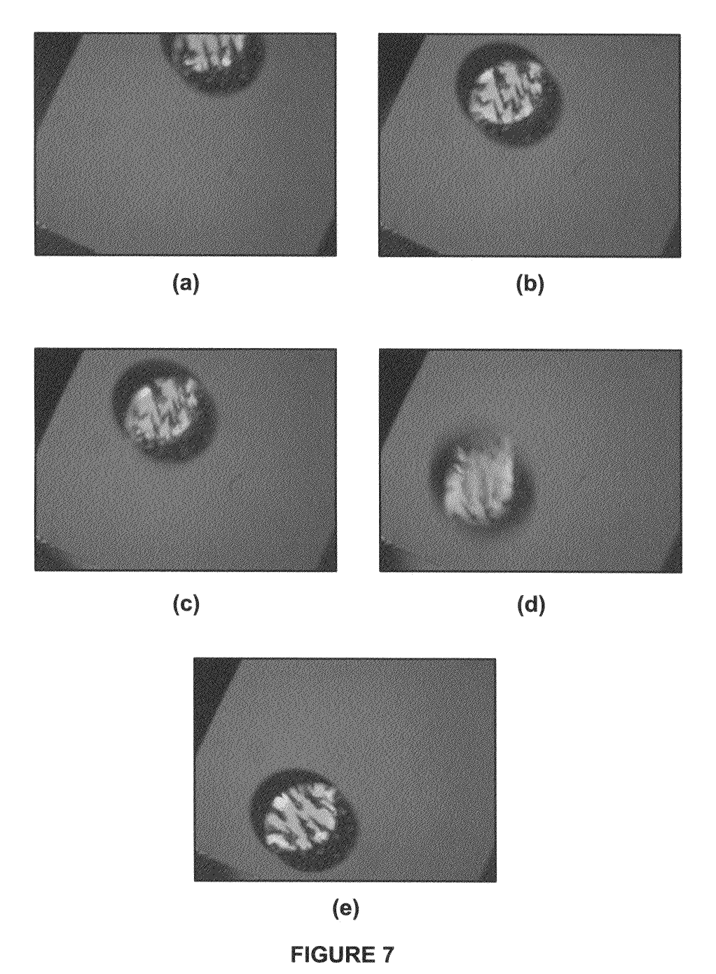

In order to investigate the mechanism by which the paramagnetic particles act on the drop and to determine how the particles were distributed inside the drop while it moved, a camera was mounted on top of the surface to record particle distribution inside a moving drop. The aggregated particle chains inside the drop were regularly distributed on the bottom of the drop as it was moving, apparently sliding with it. (See FIG. 7)

2. Coalescence

One method relates to controlling droplets of fluids on superhydrophobic surfaces through the use of magnetic fields; specifically, droplets can be combined or coalesced. In one aspect, the method further comprises the acts of positioning an additional fluid droplet in contact with the superhydrophobic surface; varying the magnetic field intensity so as to move the magnetically active fluid droplet substantially toward the additional fluid droplet; and contacting the magnetically active fluid droplet with the additional fluid droplet with a force sufficient to overcome surface tension of the magnetically active fluid droplet or the additional fluid droplet, thereby coalescing the droplets.

In a further aspect, the second fluid droplet comprises a magnetically active fluid. In a further aspect, the second fluid droplet comprises at least one of a biologically active agent or a pharmaceutically active agent or a mixture thereof. In a further aspect, the second fluid droplet comprises particles. In a further aspect, the second fluid droplet comprises paramagnetic particles. In a further aspect, the paramagnetic particles comprise functionalization. In a further aspect, the functionalization comprises at least one of a molecular recognition moiety, an optical tag, an acidic moiety, a basic moiety, a cationic moiety, and anionic moiety, a hydrophilic moiety, a hydrophobic moiety, or a stimulus-responsive molecule or a mixture thereof.

In a yet further aspect, one or more of the droplets can optionally further comprise one or more reactive components, for example, at least one of a biologically active agent, a pharmaceutically active agent, a chemically active agent, a chemical labeling agent, or a radioactive agent or a mixture thereof. Coalescing the droplets consequently mixes the components of the drops. This aspect, therefore, provides procedures for carrying out chemical reactions using digital microfluidic methods. For example, a first droplet further including a first reactive component (e.g., an activated carboxylic acid) can be coalesced with a second droplet further including a second reactive component (e.g., an amine) using the digital microfluidic methods. Upon coalescence, the combined droplet comprises both the first and second reactive components, allowing them to react and form a product (e.g., an amide). It is contemplated that additional additives (e.g., catalysts, buffers, or indicators) can also be added to the droplets to facilitate the reactions. It is also contemplated that such digital microfluidic methods can be used in automated processes, for example, in automated peptide synthesis, in automated oligonucleotide synthesis, in automated combinatorial synthesis, or in automated analytical methods.

3. Immobilization

One method relates to controlling droplets of fluids on superhydrophobic surfaces through the use of magnetic fields; specifically, droplets can be immobilized or “pinned” on the surface. In one aspect, a method of immobilizing a fluid droplet on a surface comprises the acts of positioning a magnetically active fluid droplet in contact with a superhydrophobic surface; and coupling a stationary magnetic field with at least a portion of the droplet. In a further aspect, the fluid droplet comprises a magnetically active fluid.

In a further aspect, a method of immobilizing a fluid droplet on a surface comprises the acts of positioning a fluid droplet in contact with a surface having a more hydrophobic region and a less hydrophobic region; and contacting the droplet with the less hydrophobic region. In one aspect, the more hydrophobic surface is a superhydrophobic surface. In a further aspect, the fluid droplet comprises a magnetically active fluid.

Drops containing magnetic particles can be placed and moved on the superhydrophobic surfaces, but in order to work with drops that do not contain magnetic particles a surface defect is typically present. The surface defect can be created by physical damage or damage to the superhydrophobic chemical coating. Physical damage can be created using a sharp point such as a small needle, while the chemical coat can be removed using a laser pulse. In either case, the abrupt change in contact angle in the damaged region pins a water drop that is dropped from above this region. It has been demonstrated that the movement of a water drop containing paramagnetic particles towards a water drop held by pinning and the subsequent coalescence of the drops. This can also be accomplished using two or more water drops containing paramagnetic particles using two or more magnetic fields in order to place and/or move the drops towards each other.

While drops without paramagnetic particles that are pinned due to a surface defect cannot be moved, when combined with a drop containing paramagnetic particles or when a drop with paramagnetic particle is placed on a surface defect a magnetic field can be used to force the drop out of the defect. The utility of this action is the ability to combine the two types of drops and then continue to move the combined drop for further processing. Thus, for example, an aqueous solution to be analyzed can be combined with other drops sequentially for sample pretreatment reasons and subsequently moved to another location for analysis. Depinning takes place with only a very small amount of water left behind on the defect. Visual evidence indicates that the amount of water left on the defect depends on the size of the defect. Such depinning action with essentially all of the liquid being removed from the pinned location has not been previously described in the literature using any type of force. Without wishing to be bound by theory, it is believed that this action is performed using the surface tension of the drop as a “lever” and the paramagnetic particles as a “fulcrum.”

4. Dispensing Droplets

One method relates to controlling droplets of fluids on superhydrophobic surfaces through the use of magnetic fields; specifically, droplets can be dispensed. In one aspect, the invention relates to a method of dispensing a fluid droplet from a reservoir including the acts of positioning a fluid within a reservoir having an opening; increasing the pressure within the reservoir, thereby dispensing at least a droplet of the fluid. In a further aspect, the fluid is a magnetically active fluid.

In a further aspect, the invention relates to a method of dispensing a fluid droplet from a reservoir including the acts of positioning a magnetically active fluid within a reservoir having an opening; coupling a magnetic field with at least a portion of the fluid; and moving the magnetic field substantially away from the reservoir, thereby dispensing at least a droplet of the fluid. In one aspect, the reservoir comprises a substantially enclosed chamber.

The ability to hold water drops using surface defects as well as the ability to split water drops containing paramagnetic particles indicates the following capabilities based on electrospray and electrospinning technology as well as the well-known phenomena of droplet formation due to liquid jet instabilities. For further discussion, see Fouillet, Y, Achard, J-L, Microfluidique discrete et biotechnologie, C. R. Physics 5 (2004) 577-588, which is herein incorporated by reference. This can be important since microfluidic systems with integrated dispensing, flow, and analysis are highly desirable.

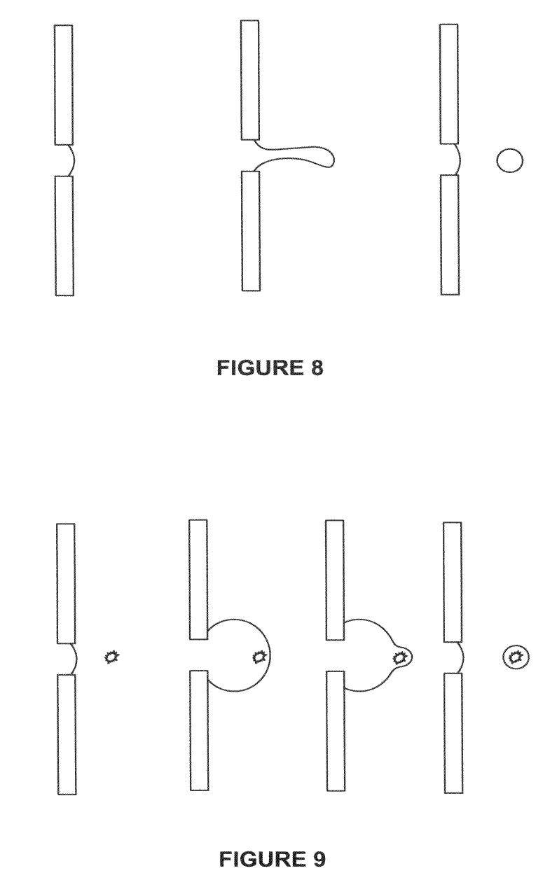

Using a simple geometric design popular in electrospray technology applied to microfluidics, drops with paramagnetic particles can be dispensed from a reservoir through the use of magnetic fields. FIG. 8 illustrates the barrier separating the hydrophilic reservoir from the superhydrophobic substrate, and shows the progression from a meniscus to an elongated drop and finally a liberated drop.

For drops that do not contain paramagnetic particles, pressure can be used to force the liquid to form a neck and a surface defect to pin the drop on the superhydrophobic substrate followed by a release of pressure. For this dispensing technique, it can be necessary to have an enclosed reservoir in order to build up sufficient pressure to force the water to enter onto the superhydrophobic surface. As shown in FIG. 9, the water drop grows relatively uniformly round in order to minimize the surface touching the superhydrophobic surface. As the radius becomes large enough to reach the surface defect shown as a star in FIG. 9, the pressure is then slowly released in order to form a neck that leads to instability followed by breakage leaving behind a drop. Based on the methods herein for combining a water drop with a drop containing paramagnetic particles, this drop can be processed and subsequently analyzed.

5. Splitting

One method relates to controlling droplets of fluids on superhydrophobic surfaces through the use of magnetic fields; specifically, a droplet can be divided or split into two or more smaller droplets. In one aspect, the invention relates to a method of dividing a fluid droplet including the acts of positioning a magnetically active fluid droplet in contact with a superhydrophobic surface; coupling a first magnetic field with at least a first portion of the droplet; coupling a second magnetic field with at least a second portion of the droplet; and varying the first magnetic field intensity so as to move the first portion substantially away from the second magnetic field with a force sufficient to overcome surface tension of the magnetically active fluid droplet, thereby dividing the first portion of the droplet from the second portion of the droplet.

6. Digital Isoelectric Focusing

One method relates to controlling droplets of fluids on superhydrophobic surfaces through the use of magnetic fields; specifically, droplets can be manipulated so as to segregate materials dissolved or dispersed within the droplet and subsequently split to divide the materials. In one aspect, the invention relates to a digital isoelectric focusing method including the acts of providing a magnetically active fluid droplet including ampholytes, a first protein having a first isoelectric point, and a second protein having a second isoelectric point different from the first isoelectric point; positioning the droplet in contact with a superhydrophobic surface; coupling an electric field with the droplet, thereby generating a pH gradient within the droplet; allowing the first protein to migrate along the pH gradient to the first isoelectric point; allowing the second protein to migrate along the pH gradient to the second isoelectric point; coupling a first magnetic field with at least a first portion of the droplet, wherein the first portion comprises the first isoelectric point; coupling a second magnetic field with at least a second portion of the droplet wherein the second portion comprises the second isoelectric point; and varying the first magnetic field intensity so as to move the first portion substantially away from the second magnetic field with a force sufficient to overcome surface tension of the magnetically active fluid droplet, thereby dividing the first portion of the droplet from the second portion of the droplet. In one aspect, the providing act is performed before the electric field is coupled with the droplet.

The methods extend digital magnetofluidics to separate proteins within a drop, based upon current practices in the use of an electric field and a group of molecules known as ampholytes in order to generate a pH gradient within a single drop. When a pH gradient is established, proteins dissolved in a droplet migrate to a particular zone in the gradient based on each of their isoelectric point. This well established process is known as isoelectric focusing (IEF) and can be done in a gel phase or in free solution.

The present invention, however, extends IEF by splitting a droplet using magnetic fields once the proteins have undergone focusing, along the longitudinal axis where the electric field is applied. Once split, one part of the former drop is enriched with a fraction of proteins above a particular isoelectric point and the other part is enriched with proteins below a particular isoelectric point. Since drops can be split and combined with the methods, this process can be repeated if further separation to more completely isolate a particular group of proteins based on isoelectric point is desired.

The choice of ampholytes can be important in IEF and DIEF. There are a number of commercially available ampholytes and special mixtures useful in particular situations. Ampholytes are well known to those of skill in the art and can be obtained commercially. Suitable ampholyte mixtures are typically low molecular weight species of different isoelectric points. The isoelectric point range can be varied by changing the chemical structure of the ampholytes. Depending on the number of different ampholytes employed and their specific isoelectric point, the pH acts and range can be altered

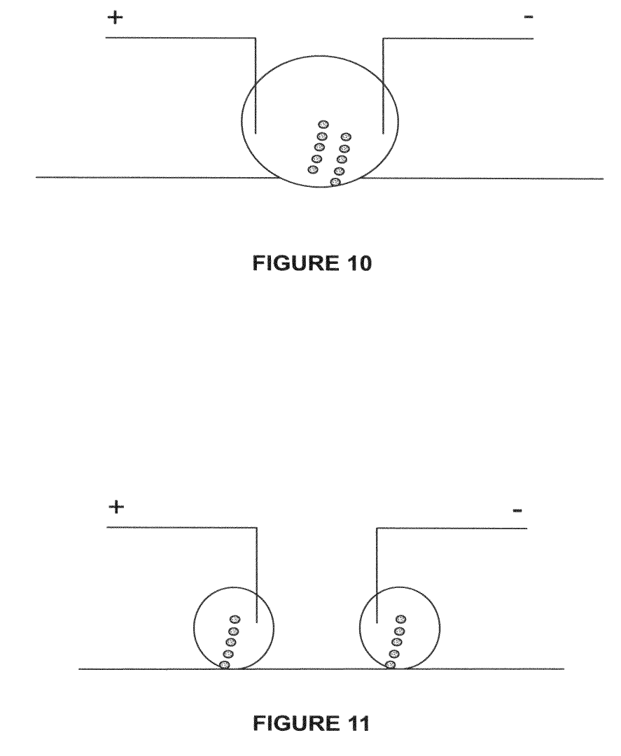

A schematic diagram of the proposed process is provided in FIGS. 10 and 11. A protein solution can be provided in a droplet. See FIG. 10. In a further aspect, another protein solution drop can be added after the electric field is applied. The droplet is subjected to an electric field, and the protein(s) migrate within the droplet as related to isoelectric point. See FIG. 11.

### E. COMBINATION METHODS

It is also contemplated that the digital magnetofluidic devices and methods can be used in combination with methods employing other forces, for example, gravity or light-driven methods.

1. Gravity Methods

In one aspect, the devices and methods can be used in combination with gravity-based methods. For example, gravity can be used to create a force across a surface at an angle other than substantially perpendicular to a gravitational field. The vector of the gravitational field can combine with a force created by a magnetofluidic vector to produce a net force on a fluid droplet.

2. Light-Driven Microfluidic Methods

In one aspect, the devices and methods can be used in combination with light-driven methods as disclosed in Rosario, R., et al., “Lotus Effect Amplifies Light-Induced Contact Angle Switching,” J. Phys. Chem. B, 2004, 108, 12640-12642, which is herein incorporated by reference. For example, a hydrophobicity gradient can be created by a functionalized surface in response to a light frequency gradient to create a net force across a hydrophobic surface.

In one aspect, the methods can be used in combination with a device including a surface, wherein the surface has roughness, a hydrophobic layer, and a photoresponsive molecule. In a further aspect, the methods can be used in combination with a device including a surface, wherein the surface has roughness, a hydrophobic layer, and an isomerization molecule which can be isomerized into a first and a second form, wherein the first and second forms have different effects on the wetting of the surface by a fluid. In a further aspect, the methods can be used in combination with a device including a fractally rough, hydrophobic surface, and a liquid droplet, wherein the liquid droplet has a contact angle with the surface, and wherein the advancing contact angle under a first condition is lower than the receding contact angle under a second condition. In a further aspect, the methods can be used in combination with a device including a surface, wherein the surface has roughness, a hydrophobic layer, and a stimulus inducible molecule, wherein the stimulus inducible molecule causes a contact angle change when stimulated, producing a stimulus induced contact angle change. In one aspect, the methods can be used in combination with a hydrophobic surface that has roughness and a hydrophobic layer. In a further aspect, the roughness is a well ordered microstructure. In a yet further aspect, the roughness is a well ordered nanostructure. In a yet further aspect, the roughness is a random fractal geometry.

In a yet further aspect, the superhydrophobic surface comprises a nanoscale structure. The nanoscale structure can be grown by, for example, one or more of a vapor-liquid-solid technique, a chemical or physical vapor deposition onto patterned substrates, dry plasma deposition of pattered substrates, wet etching of a patterned substrate, or deposition of separately fabricated nanostructured materials. In a further aspect, the separately fabricated nanostructured materials are nanodots or nanowires.

A. Nanowires

In a further aspect, the nanoscale structure comprises a nanowire. In a yet further aspect, the nanowire comprises at least one magnetically active material or at least one magnetically inactive material. In a further aspect, the nanowire comprises silicon, zinc oxide, alumina, silicon dioxide, titanium, tungsten, tantalum, iron, nickel, or alloy nanowire or a mixture thereof. In a further aspect, the nanowire comprises a silicon nanowire. In a further aspect, the nanowire is in one or more of a random array of nanowires, an ordered array of nanowires, or a hierarchically patterned array of nanowires. In one aspect, the device comprises a nanowire having a diameter of from about 1 nm to about 100 micrometers, from about 10 nm to about 100 micrometers, from about 10 nm to about 200 nm, from about 20 nm to about 500 nm, from about 20 nm to about 100 nm, or from about 20 nm to about 50 nm.

B. Hydrophobic Layer

In one aspect, the device comprises a superhydrophobic layer including a hydrocarbon. In a further aspect, the superhydrophobic layer comprises a perfluorinated hydrocarbon. In a further aspect, the superhydrophobic layer further comprises at least one stimulus-responsive molecule. In various aspects, the stimulus can comprise at least one of light, heat, pH, a biologically active molecule, or solution chemistry or a combination thereof.

C. Photoresponsive Molecules

Photoresponsive molecules, or stimulus inducible/responsive molecules, or variable hydrophobicity molecules, can be used to create a hydrophobicity gradient in response to a light frequency gradient to create a net force across a superhydrophobic surface.

In one aspect, the stimulus-responsive molecule comprises an isomerization molecule which can be isomerized between a first form and a second form, wherein the first form and second form have different effects on the wetting of the surface. In a further aspect, the stimulus-responsive molecule comprises an isomerization molecule which can be isomerized between a first form and a second form, wherein the first form is more hydrophilic than the second form. In a further aspect, the stimulus-responsive molecule comprises an isomerization molecule which can be isomerized between a first form and a second form, wherein the first form is more polar than the second form. In a further aspect, the stimulus-responsive molecule has predominantly a polar form when exposed to light having a first wavelength. In a further aspect, the stimulus-responsive molecule has predominantly a nonpolar form when exposed to light having a second wavelength.

In one aspect, the stimulus-responsive molecule is a photochrome. In a further aspect, the photochrome isomerizes under two different wavelengths of light. In a further aspect, the photochrome comprises an organic molecule. In a further aspect, the photochrome is covalently attached to the surface. The photochrome can be, for example, one or more of a spiropyran, an indolinospiropyran, a spirooxazine, a benzo-naphthopyran, a naphthopyran, an azobenzene, a fulgide, a diarylethene, a dihydroindolizine, a photochromic quinone, a perimidinespirocyclohexadienone, or a dihydropyrene or a combination thereof.

(1) Spiropyrans

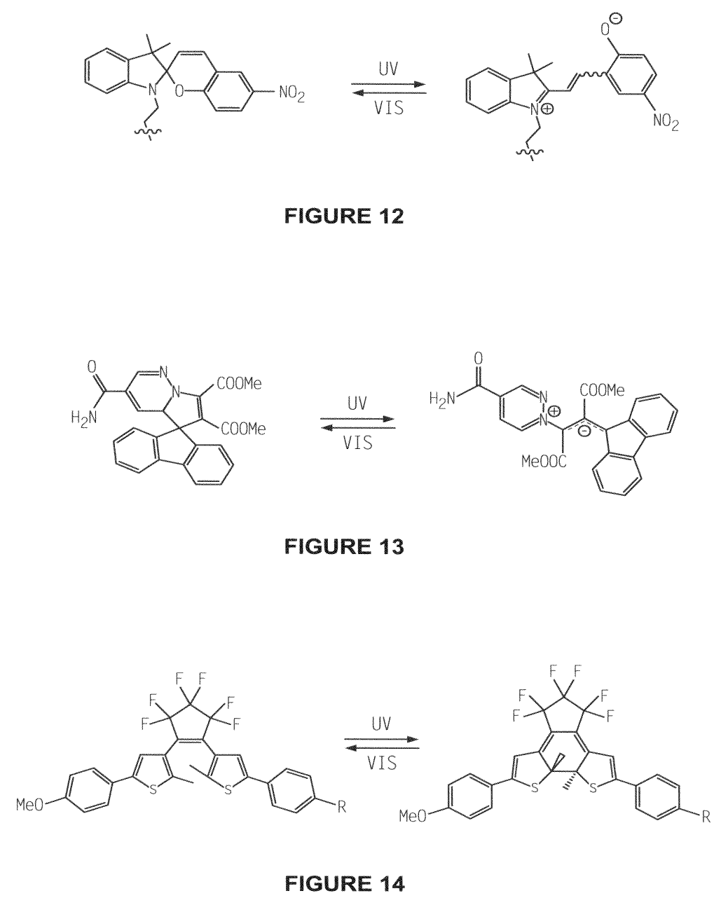

Spiropyrans are a class of organic photochromes that undergo a reversible transition from a closed, nonpolar form to a highly polar, open form when irradiated with higher energy, shorter wavelength light (e.g., ultraviolet (UV) light (e.g., 366 nm)). (FIG. 12).

Irradiation with lower energy, longer wavelength light (e.g., visible (VIS) light (e.g., 450-550 nm)) converts the molecule back to its closed, nonpolar form. Visible light irradiation of the spiropyran coating yields a relatively hydrophobic surface (higher contact angle) that can be reversibly converted into a more hydrophilic surface (lower contact angle) with UV light irradiation. The reversible switching of contact angles using UV and visible light for these molecular monolayers on smooth glass surfaces is due to the photon-modulated conversion of the spiropyran molecules between open and closed forms. For further discussion, see Rosario, R. et al., Photon-modulated wettability changes on spiropyran-coated surfaces, Langmuir 18, 8062-8069 (2002), which is herein incorporated by reference.

(2) Dihydroindolizines

Dihydroindolizines are a class of organic photochromes that undergo a reversible transition from a closed, nonpolar, form to a highly polar, open form when irradiated with higher energy, shorter wavelength light (e.g., ultraviolet (UV) light). (FIG. 13).

(3) Dithienylethene

Dithienylethenes are a class of organic photochromes that undergo a reversible transition from an open, nonplanar form to a closed, planar form when irradiated with higher energy, shorter wavelength light (e.g., ultraviolet (UV) light). (FIG. 14).

(4) Dihydropyrene

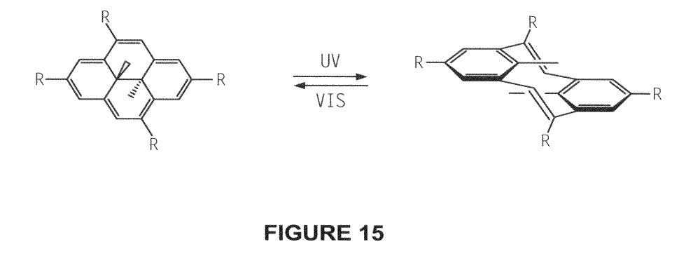

Dihydropyrenes are a class of organic photochromes that undergo a reversible transition from a closed, planar form to an open, nonplanar form when irradiated with higher energy, shorter wavelength light (e.g., ultraviolet (UV) light). (FIG. 15).

### F. KITS

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

### G. COMPOSITIONS WITH SIMILAR PURPOSES

It is understood that the compositions disclosed herein have certain purposes. Disclosed herein are certain structural requirements for performing the disclosed purposes, and it is understood that there are a variety of structures which can perform the same purpose which are related to the disclosed structures, and that these structures will ultimately achieve the same result.

### H. METHODS OF MAKING THE COMPOSITIONS AND DEVICES

The compositions disclosed herein and the compositions necessary to perform the disclosed methods can be made using any method known to those of skill in the art for that particular reagent or compound unless otherwise specifically noted.

Disclosed are processes for making the compositions and devices as well as making the intermediates leading to the compositions. There are a variety of methods that can be used for making these compositions, such as synthetic chemical methods and standard molecular biology methods. It is understood that the methods of making these and the other disclosed compositions are specifically disclosed.

### I. METHODS OF USING THE COMPOSITIONS

The disclosed compositions can be used in a variety of ways as research tools. The compositions can be used, for example, in screening protocols to isolate molecules that possess desired properties.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

### J. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Preparation of Superhydrophobic Surfaces

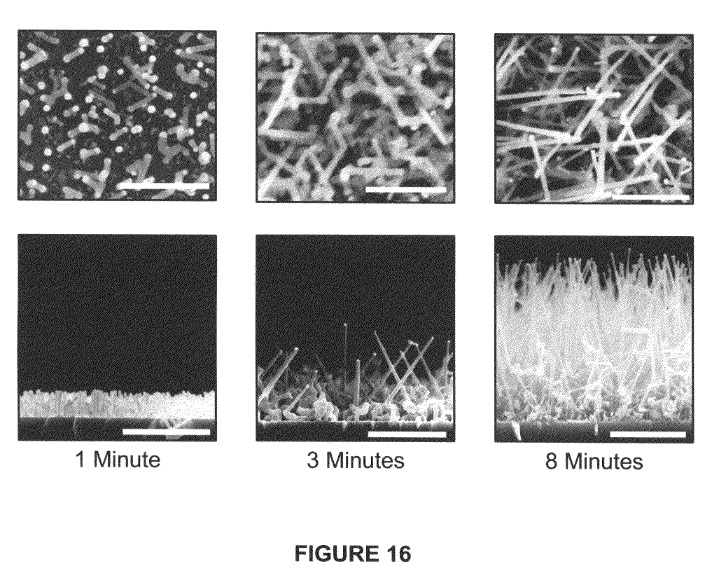

Superhydrophobic surfaces were prepared using vapor-liquid-solid (VLS) growth systems to create high aspect ratio Si nanowires with various diameters, spacing, and lengths. In order to create the hydrophobic effect, a perfluoronated hydrocarbon coating was covalently applied to the entire nanowire surface. The resultant superhydrophobic nanowire surfaces do not follow a simple geometric pattern and exhibit fractal, multidimensional, random roughness, with contact angles near 180 degrees. For further discussion, see Dailey, J. W., et al., Vapor-liquid-solid growth of germanium nanostructures on silicon. Journal of applied physics, 2004. 96(12): p. 7556-7567, which is herein incorporated by reference. The VLS growth technique employs small dots of gold that act as catalytic seeds for growing a high density of nanowires on a surface (FIG. 16). During evaporation of a few monolayers of Au on a clean Si or glass surface, the Au self assembles into nanodots. In the subsequent VLS synthesis the Au dots form a eutectic liquid with Si from which liquid-mediated growth of single crystal Si nanowires occurs. The nanowire diameters are set by the Au dot diameters, with one-dimensional growth occurring as the AuSi eutectic dot rides along at the free end of the growing wire. The growth rate is linear in time and pressure, and the length of the nanowires is thus easily controlled by fixing the growth time. Typically, the Au dots at the end of the nanowires account for only a very small area. If desired, they can be chemically removed after growth to eliminate any effect they may have on interfacial properties.

2. Preparation of Magnetically Active Fluid droplets

A small amount of paramagnetic particles was added to water drops, which were placed and held over a superhydrophobic surface sample by a permanent magnet located below the surface (FIG. 1). Milli-Q water was used to prepare aqueous solution with different particle concentrations ranging from 0.1% to 5%. The spherical paramagnetic carbonyl iron particles used were acquired from Lord Corporation. The particles are highly polydisperse in size (ranging from 0.2 to 4.0 μm) and they have a high saturation magnetization (211 emu/g). The magnetic field was generated by a NdFeB bar magnet located below the superhydrophobic surface. Drop movement was studied by recording images with two CCD cameras provided with zoom systems (Navitar 12×). One camera was located at one side of the drop and the other camera above the drop

3. Effect of Composition and Viscosity of Droplets

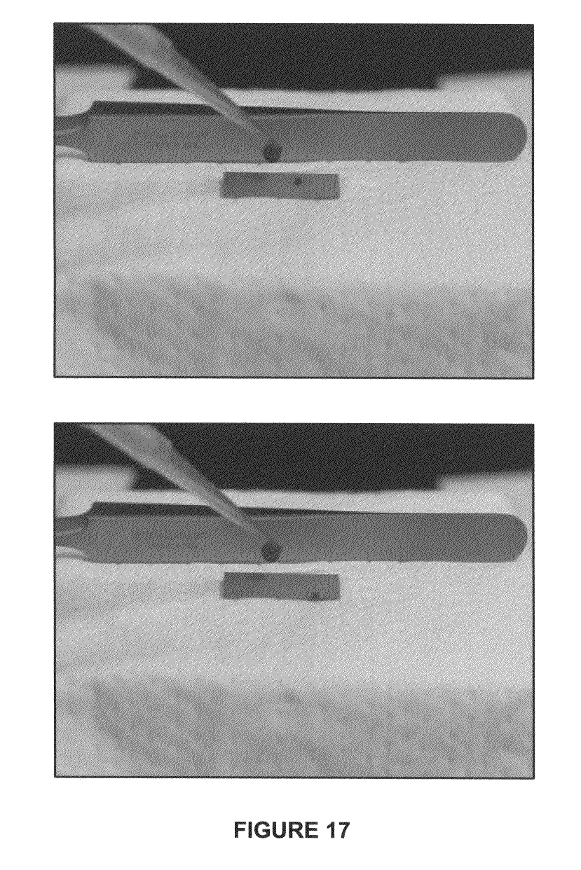

The movement of blood droplets was observed and recorded using a digital camera on a superhydrophobic surface. These blood droplets, at first, appeared to roll off. Then, after observing several droplets, the droplets behaved differently-appearing to stick. Simply lifting one end of the material, however, allowed the droplets to slide off (See FIG. 17).

It is possible that blood viscosity may have prevented the droplets from sliding off. Notably, the heparinized blood appeared to coagulate during the course of the blood droplet study. It will be interesting to investigate less viscous blood solutions in order to understand how viscosity affects droplet movement on these superhydrophobic surfaces.

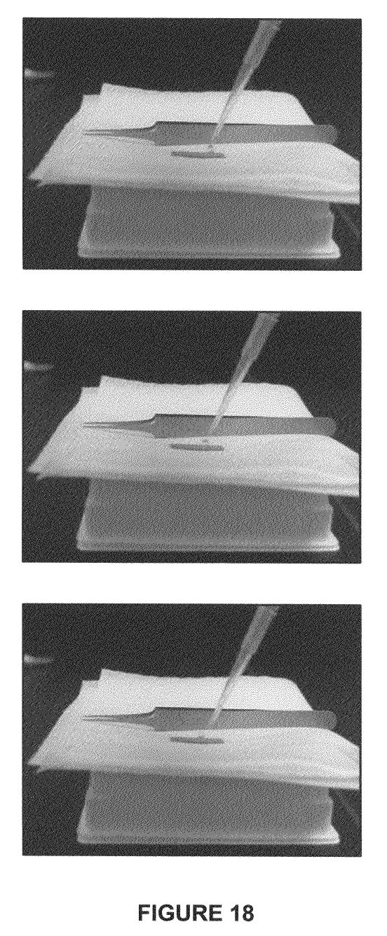

This blood droplet study was followed by observations of urine droplet movement. See FIG. 18. These urine droplets behaved like water-easily sliding off the surface.

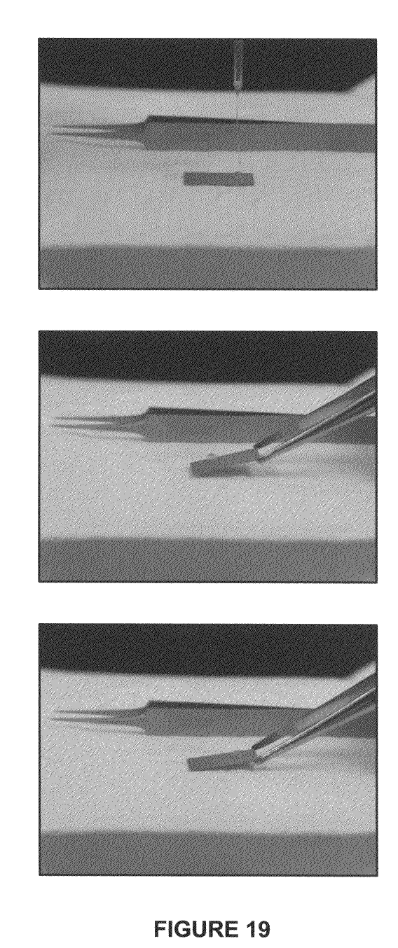

Saliva, plasma, and serum droplets were also observed on the superhydrophobic surface. See FIG. 19. These droplets were very difficult to deliver using a pipette due to their viscosity and overall stickiness. Once deposited, the droplets tended to stick, but could be moved by elevating one end of the surface. At an angle, the droplets rolled off the surface.

4. Coalescence of Droplets

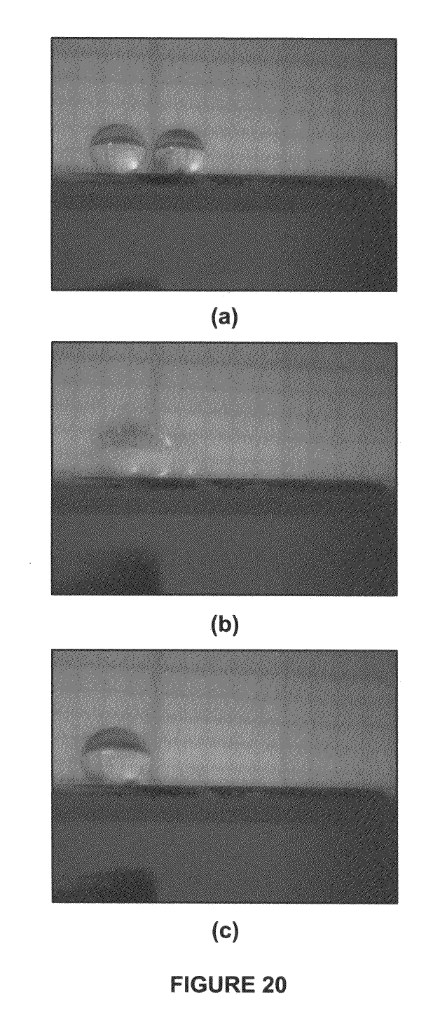

Coalescence of two drops was achieved by placing a 6 microliter water drop without particles deliberately on a surface defect to hold it. Another 4 microliter water drop containing paramagnetic particles was displaced over the surface by the action of the magnet towards the pure water drop, until they were close enough to touch and coalesce. After coalescence, the combined drop was pulled out of the surface defect by the magnet. (See FIG. 20)

5. Splitting of Droplets

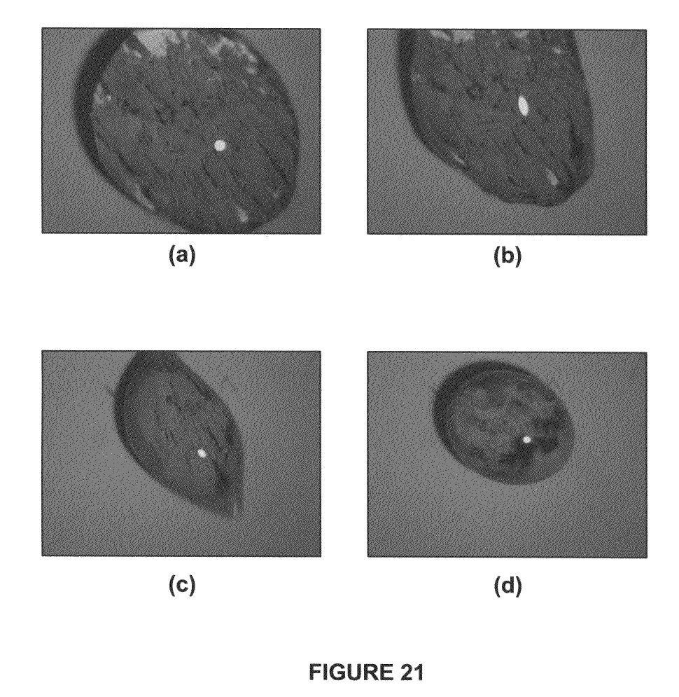

In a drop splitting experiment, a drop was loaded with a high concentration of paramagnetic particles and two magnets were placed below the surface of the drop. The drop spread under the influence of the separating magnets until it split (See FIG. 21).

6. Movement of Liquid by Light-Induced Changes

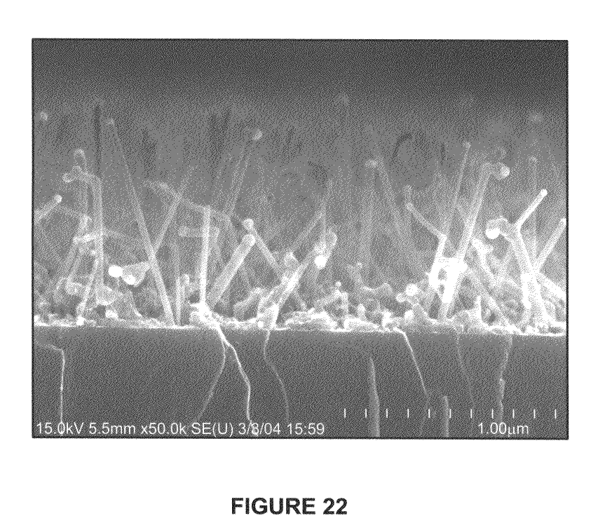

A polished silicon wafer bearing random silicon nanowires with diameters of 20-50 nm was prepared by a vapor-liquid-solid technique. For further discussion, see Wagner, R. S. in Whisker Technology (ed. Levit, A. P.) 47-119 (Wiley-Interscience, New York, 1970), which is herein incorporated by reference. (FIG. 22) The air-oxidized silicon surface was treated with tert-butyldiphenylchlorosilane and perfluorooctyltrichlorosilane, followed by 3-aminopropyldiethoxymethylsilane, to which a photochromic spiropyran molecule was later attached by a published technique. For further discussion, see Rosario, R. et al., Photon-modulated wettability changes on spiropyran-coated surfaces, Langmuir 18, 8062-8069 (2002), which is herein incorporated by reference.

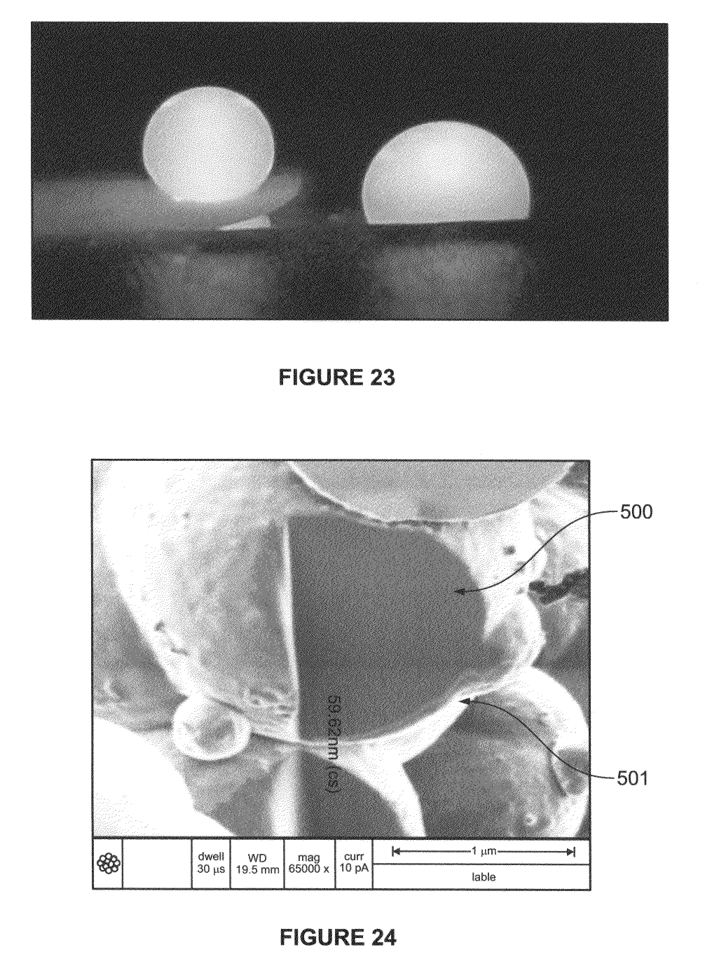

After surface derivatization with spiropyran-containing monolayers on silicon (Si) nanowire and adjacent smooth silicon surfaces, multiple measurements of advancing and receding water contact angles under UV and visible irradiation were performed using the sessile drop method. Direct comparisons of water contact angles on adjacent polished and nanowire areas are shown in FIG. 23. The combination of the surface roughness and the superhydrophobic coating resulted in significantly higher contact angles on the nanowire surface compared to the smooth surface.

The average advancing contact angle on the smooth surface was 12° lower under UV irradiation than under visible irradiation (FIG. 4). On the nanowire-bearing surface, this light-induced contact angle change increased to 23° (FIG. 4). The increase in the light-induced contact angle changes on the nanowire-bearing surface confirmed that roughness has the effect of amplifying stimulus-induced contact angle changes relative to smooth surfaces by nearly a factor of two.

Under visible irradiation of the spiropyran-coated surfaces, the water contact angle hysteresis was measured to be 37° on the smooth surface, whereas on the nanowire-bearing surface a significantly lower value of only 17° was observed.

In a control experiment on the smooth spiropyran-coated surface, the advancing water contact angle under UV irradiation (110°) was higher than the receding water contact angle under visible irradiation (85°). This does not fulfill the criterion for liquid motion, and it was found that water drops on the smooth surface could not be moved using light.

In contrast, on the spiropyran-coated nanowire-bearing surface, the advancing water contact angle under UV irradiation (133°) was lower than the receding water contact angle under visible irradiation (140°). Accordingly, when an ultraviolet light-visible light gradient was applied across water drops sitting on the nanowire-bearing surface, the drops moved towards the UV end of the gradient.

Control experiments performed on drops sitting on nanowire-bearing surfaces coated with the superhydrophobic layer, but without the spiropyran, did not result in any drop motion. Therefore, it can be concluded that the motion of the water droplets on the photoresponsive, nanowire-bearing surface was due to the roughness-magnified light-induced switching of surface energy by the spiropyrans coupled with the lower contact angle hysteresis of the superhydrophobic surface.

Thus, it is demonstrated that surface roughness can be an effective tool for the amplification of stimulus-induced contact angle switching. The degree of amplification due to roughness was predicted using a Wenzel model. The combination of roughness-amplification of contact angle change with the reduced contact angle hysteresis of the nanowire-bearing, photoresponsive surfaces resulted in advancing contact angles under UV irradiation that were lower than the receding angles under visible irradiation. This for the first time permitted water drops on the nanowire surface to be moved solely using gradients of UV and visible light.

This result can lead to the development of photonic control of water movement in microfluidic devices. Additionally, since the fluid driving force in electrowetting (as described in Lahann, J. et al., A reversibly switching surface, Science 299, 371-374 (2003); Schneemilch, M., Welters, W. J., Hayes, R. A. & Ralston, J., Electrically induced changes in dynamic wettability. Langmuir 16, 2924-2927 (2000), both of which are herein incorporated by reference) and thermowetting (as described in Yakushiji, T. & Sakai, K., Graft architectural effects on thermoresponsive wettability changes of poly(N-isopropylacrylamide)-modified surfaces, Langmuir 14, 4657-4662 (1998); Liang, L., Ski, M., Viswanathan, V. V., Peummg, L. M. & Young, J. S., Temperature-sensitive polypropylene membranes prepared by plasma polymerization, Journal of Membrane Science 177, 97-108 (2000), both of which are herein incorporated by reference) microfluidic systems is also the stimulus-induced difference between advancing and receding contact angles, these findings can enhance fluidic motion and control in these systems.

7. Hydrophobic/Spiropyran Coating Procedure

Both nanowire and flat silicon oxide samples were cleaned using a 1:1 volume ratio of methanol/concentrated hydrochloric acid solution, followed by extensive washing in deionized water, yielding a nanowire surface contact angle of about 0° and a flat surface contact angle of 19°. The samples were then treated with a toluene solution of tert-butyldiphenylchlorosilane and perfluorooctyltrichlorosilane in the ratio of 10:1, giving a nanowire surface contact angle of about 175° (i.e., Cassie-Baxter) and a flat surface contact angle of 106±2°. This was followed by treatment with a toluene solution of (3-aminopropyl)diethoxymethylsilane and curing at 140° C., yielding a nanowire surface contact angle of about 175° and a flat surface contact angle of 103±3°. The silane-treated nanowire and flat silicon oxide samples were then incubated in an ethanolic solution of a photochromic spiropyran acid (1 mM) in the presence of the coupling agent 1-ethyl-3-(3-(dimethylamino)propyl)carbodiimide (10 mM), washed sequentially with ethanol and water, and dried under vacuum, producing a nanowire surface contact angle of about 174° and a flat surface contact angle of 107±8° under visible irradiation.

A. Growth of Nanowires

Silicon nanowires were prepared by a vapor-liquid-solid (VLS) growth technique, using small dots of gold that act as catalytic seeds for growing a high density of nanowires on silicon substrates (FIG. 3). During evaporation of a few monolayers of Au on a clean Si or glass surface, the Au self assembles into nanodots. In the subsequent VLS synthesis the Au dots form a eutectic liquid with Si from which liquid-mediated growth of single crystal Si nanowires occurs. The nanowire diameters are set by the Au dot diameters, with one-dimensional growth occurring as the AuSi eutectic dot rides along at the free end of the growing wire. The growth rate is linear in time and the length of the nanowires is thus easily controlled by fixing the growth time. The Au dots at the end of the nanowires account for only a very small area. Typical VLS silicon nanowire growth conditions for these studies were 400 to 650° C. with disilane gas pressures of 3-500 mTorr, resulting in nanowire diameters of 20-100 nm and lengths of 1-3 μm.

B. UV-Ozone Treatment

In one approach to the study of the effect of surface chemistry changes on nanowire surfaces without altering the surface geometry, a UV-ozone cleaner (Jelight Company Inc., model 42) was used. This apparatus contains a UV source and a chamber with adjustable oxygen flow and pressure. Atomic oxygen is generated when molecular oxygen and ozone are dissociated by UV light. Any organic coating on the nanowires reacts with atomic oxygen, forming volatile molecules that desorb from the surface. The process is known not to damage delicate structures in semiconductor processing. A nanowire coating can thus be removed to different degrees, leading to a continuous variation in hydrophobicity, by varying the treatment time while conducting the cleaning at room temperature.

C. Contact Angle Measurements

Advancing and receding contact angle measurements were performed using a Rame-Hart Model 250 standard automated goniometer. For measuring the advancing angle on flat surfaces, 5 microliters of deionized water was dropped onto the sample from a microsyringe bearing a needle with a hydrophobic tip. For superhydrophobic surfaces, a larger drop of about 15-20 microliters was used because smaller drops easily rolled off the surface. This led to a small degree of measurement error since the drop was not fully spherical. An image of the drop was taken shortly after the drop was deposited in order to avoid measurement error due to drying. For receding angles, the microsyringe needle was used to draw some of the water out of the drop. The software automatically generates tangent measurements on the drop profiles. Usually four measurements were taken on different parts of the sample surface in order to characterize the overall properties of the surface.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### K. COATING THE MAGNETIC PARTICLES

In various representative aspects, the invention described here uses coated magnetic, ferromagnetic, or paramagnetic particles to move drops in digital magnetofluidics. This coating is very thin and may protect the magnetically active particles from oxidation or reaction with water or chemicals in the fluid surrounding them. Essentially, the coating may form an impermeable barrier on the surface of magnetically active particles.

In one representative aspect, aqueous drops containing paramagnetic iron particles (Sigma-Aldrich, Inc., St. Louis, Mo.) were pipetted onto a superhydrophobic surface. The iron particles were coated with polysiloxane. Iron-polysiloxane composites were prepared by hydrolysis-condensation polymerization of tetraorthosilicate (Sigma-Aldrich, Inc., St. Louis, Mo.). Briefly, iron particles (20 g) were added to a mixture of tetraorthosilicate (40 mL) and ethyl alcohol (160 mL) and stirred. Next, 10 mL of ammonium hydroxide (25 wt percent; Sigma-Aldrich, Inc., St. Louis, Mo.) was slowly added to the mixture, which was them stirred for 24 h. at room temperature. Coated particles were washed three times with ethyl alcohol, four times with deionized water, and dried at 60° C. in a vacuum over for 24 h.

### L. EXAMPLE ASPECTS

In one aspects, a digital magnetofluidic device includes a superhydrophobic surface, a magnetically active fluid bead comprising a magnetically active substrate enclosed by a coating and a fluid drop formed around the coated substrate, and a magnetic field coupled with at least a portion of the magnetically active fluid bead.

This and other aspects can include one or more of the following features. The coating can be an anti-fouling layer. The coating can include polysiloxane. The magnetic field can have a strength of at least about 0.05 nN, at least about 0.1 nN, at least about 0.2 nN, at least about 0.3 nN, at least about 0.4 nN, at least about 0.5 nN, at least about 0.6 nN, at least about 0.7 nN, at least about 0.8 nN, at least about 0.9 nN, at least about 1 nN, about 0.1 nN, about 0.2 nN, about 0.3 nN, about 0.4 nN, about 0.5 nN, about 1 nN, about 2 nN, about 5 nN, or about 10 nN. The magnetic field can be produced by a permanent magnet or an electromagnet. The magnetic field can rotating. The fluid drop can be an aqueous fluid drop. The aqueous fluid can include at least one of water, sea water, freshwater, wastewater, saliva, blood, semen, plasma, urine, lymph, serum, tears, vaginal fluid, sweat, plant or vegetable extract fluid, or cell or tissue culture media, or a mixture thereof. The drop can include at least one of a biologically active agent or a pharmaceutically active agent or a mixture thereof. The fluid drop can include ampholytes. The fluid drop can include at least one of a chemically active agent, a chemical labeling agent, or a radioactive agent or a mixture thereof.

An electric field can be coupled with at least a portion of the magnetically active substrate. The magnetically active substrate can include at least one of a paramagnetic material, a diamagnetic material, or a ferromagnetic material or a mixture thereof. The at least one of a paramagnetic material, a diamagnetic material, or a ferromagnetic material or a mixture thereof can be present in the fluid bead at a concentration of from about 0.05% (w/v) to about 5% (w/v), from about 0.1% (w/v) to about 10% (w/v), from about 0.5% (w/v) to about 5% (w/v), from about 1% (w/v) to about 10% (w/v), or from about 0.1% (w/v) to about 1% (w/v).

The magnetically active substrate can include a paramagnetic particle. The paramagnetic particle can be functionalized. The functionalization can include at least one of a molecular recognition moiety, an optical tag, an acidic moiety, a basic moiety, a cationic moiety, and anionic moiety, a hydrophilic moiety, a hydrophobic moiety, or a stimulus-responsive molecule or a mixture thereof. The magnetically active substrate can include at least one of iron, nickel, or cobalt or a mixture thereof. The magnetically active substrate can include at least one paramagnetic carbonyl iron particle.

The magnetically active fluid bead can have a contact angle with the superhydrophobic surface. The contact angle can be magnified relative to a smooth surface. The magnetically active fluid bead can be in motion across the surface of the superhydrophobic surface, thereby creating an advancing edge contact angle and a receding edge contact angle. The magnetically active fluid bead can have a contact angle hysteresis that is decreased relative to a smooth surface.

The contact angle between the magnetically active fluid bead and the superhydrophobic surface can be at least about 120°, at least about 130°, at least about 140°, at least about 150°, at least about 155°, at least about 160°, or at least about 165°. The contact angle between the magnetically active fluid bead and the superhydrophobic surface can be from about 120° to about 180°, from about 130° to about 180°, from about 140° to about 180°, from about 150° to about 180°, from about 155° to about 180°, from about 160° to about 180°, from about 165° to about 180°, from about 140° to about 160°, from about 150° to about 170°, or about 160°.

The superhydrophobic surface can include at least two regions of differing hydrophobicity. For example, the superhydrophobic surface can include a wettability gradient. The superhydrophobic surface can include at least two different superhydrophobic materials having differing superhydrophobicities. The superhydrophobic surface can include at least two superhydrophobic materials having differing roughnesses. The superhydrophobic surface can include poly(tert-butyl acrylate)-block-poly(dimethylsiloxane)-block-poly(tert-butyl acrylate). The superhydrophobic surface can include superhydrophobic isotactic polypropylene. The superhydrophobic surface can include superhydrophobic boehmite or superhydrophobic silica. The superhydrophobic surface can include a superhydrophobic fluorine-containing nanocomposite coating prepared from a sol gel prepared from tetraethoxysilane, 1H,1H,2H,2H-perfluorooctyltriethoxysilane, and silica. The superhydrophobic surface can include polytetrafluoroethylene coated mesh film. The superhydrophobic surface can include fluorinated dislocation-etched aluminum. The superhydrophobic surface can include a multiplicity of carbon nanotubes. The superhydrophobic surface can include a multiplicity of carbon nanotubes coated with polytetrafluoroethylene. The superhydrophobic surface can include a multiplicity of carbon nanotubes coated with a zinc oxide thin film. The superhydrophobic surface can include a multiplicity of superhydrophobic amphiphilic poly(vinyl alcohol) nanofibers. The superhydrophobic surface can include anode oxidized aluminum. The superhydrophobic surface can include a superhydrophobic coating comprising residues of 1H,1H,2H,2H-perfluorooctyltrichlorosilane or 1H,1H,2H,2H-perfluorodecyltrichlorosilane. The superhydrophobic surface can include a superhydrophobic micropatterned polymer film having micro- or nano-scale surface concavities. The superhydrophobic surface can include a superhydrophobic porous poly(vinylidene fluoride) membrane. The superhydrophobic surface can include superhydrophobic microstructured zinc oxide. The superhydrophobic surface can include conductive superhydrophobic microstructured zinc oxide. The d superhydrophobic surface can include a superhydrophobic block copolymer of polypropylene and poly(methyl methacrylate). The superhydrophobic surface can include a superhydrophobic block copolymer of fluorine-end-capped polyurethane and poly(methyl methacrylate). The superhydrophobic surface can include superhydrophobic low-density polyethylene. The superhydrophobic surface can include a superhydrophobic film deposited by microwave plasma-enhanced chemical vapor deposition of trimethyltrimethoxysilane and carbon dioxide. The superhydrophobic surface can include a superhydrophobic polystyrene microsphere/nanofiber composite film. The superhydrophobic surface can include a superhydrophobic coating comprising residues of 2-(3-(triethoxysilyl)propylaminocarbonylamino)-6-methyl-4[1H]pyrimidinone. The superhydrophobic surface can include a superhydrophobic calcium carbonate and poly(N-isopropyl acrylamide) hierarchical structure. The superhydrophobic surface can include superhydrophobic electrospun polystyrene trichomelike structures. The superhydrophobic surface can include a superhydrophobic copolymer comprising poly((3-trimethoxysilyl)propyl methacrylate-r-polyethylene glycol methyl ether methacrylate). The superhydrophobic surface can include microscale features produced by sol-gel etching. The superhydrophobic surface can include roughness and a superhydrophobic layer. The roughness can be a well ordered microstructure and/or a random fractal geometry.

The superhydrophobic surface can include a nanoscale structure. The nanoscale structure can be grown by a vapor-liquid-solid technique, by a chemical or physical vapor deposition onto patterned substrates, by dry plasma deposition of pattered substrates, by wet etching of a patterned substrate, or by deposition of separately fabricated nanostructured materials. The nanoscale structure can be grown by a vapor-liquid-solid technique. The separately fabricated nanostructured materials are nanodots or nanowires. The nanoscale structure can include a nanowire. The nanowire can include at least one magnetically active material. The nanowire can include at least one magnetically inactive material. The nanowire can include silicon, zinc oxide, alumina, silicon dioxide, titanium, tungsten, tantalum, iron, nickel, or alloy nanowire or a mixture thereof. The nanowire can include a silicon nanowire. The nanowire can be in one or more of a random array of nanowires, an ordered array of nanowires, or a hierarchically patterned array of nanowires. The nanowire can have a diameter of from about 1 nm to about 100 micrometers, from about 10 nm to about 100 micrometers, from about 10 nm to about 200 nm, from about 20 nm to about 500 nm, from about 20 nm to about 100 nm, or from about 20 nm to about 50 nm.

The superhydrophobic layer can include a hydrocarbon, such as a perfluorinated hydrocarbon. The superhydrophobic layer can include at least one stimulus-responsive molecule. For example, the stimulus can include at least one of light, heat, pH, a biologically active molecule, or solution chemistry or a combination thereof. The stimulus-responsive molecule can include an isomerization molecule which can be isomerized between a first form and a second form. The first form and second form can have different effects on the wetting of the surface. The stimulus-responsive molecule can include an isomerization molecule which can be isomerized between a first form and a second form. The first form can be more hydrophilic than the second form. The stimulus-responsive molecule can include an isomerization molecule which can be isomerized between a first form and a second form. The first form can be more polar than the second form. The stimulus-responsive molecule can have a predominantly polar form when exposed to light having a first wavelength and a predominantly nonpolar form when exposed to light having a second wavelength. The stimulus-responsive molecule can be a photochrome. The photochrome can isomerize under two different wavelengths of light. The photochrome can include an organic molecule. The photochrome can be covalently attached to the surface. The photochrome can be a spiropyran such as, e.g., an indolinospiropyran. The photochrome can include a spirooxazine, benzo-naphthopyran, naphthopyran, azobenzene, fulgide, diarylethene, dihydroindolizine, photochromic quinone, perimidinespirocyclohexadienone, or dihydropyrene or a combination thereof.

In another aspect, a method of inducing linear movement of a fluid bead on a superhydrophobic surface includes positioning a magnetically active fluid bead that includes a magnetically active substrate enclosed by a coating and a fluid drop formed around the coated substrate, in contact with a superhydrophobic surface, coupling a magnetic field with at least a portion of the fluid bead, and varying the magnetic field intensity across the surface.

This and other aspects can include one or more of the following features. The magnetic field can have an intensity sufficient to overcome friction between the magnetically active fluid bead and the superhydrophobic surface but insufficient to overcome the surface tension of the magnetically active fluid bead. The coating can include an anti-fouling layer. The coating can include polysiloxane.

The magnetic field can have an intensity of about 0.1nN or an intensity of about in N. The superhydrophobic surface can include at least one stimulus-responsive molecule. The magnetic field can be varied so as to produce a bead speed of about 0.5 cm/s, about 1 cm/s, about 2 cm/s, about 3 cm/s, about 4 cm/s, about 5 cm/s, about 6 cm/s, or about 7 cm/s. The method can include rotating the magnetic field. The magnetically active fluid bead can include at least one of a biologically active agent or a pharmaceutically active agent or a mixture thereof. The magnetically active fluid bead can include at least one of a chemically active agent, a chemical labeling agent, or a radioactive agent or a mixture thereof. The magnetically active substrate can include a paramagnetic particle. The paramagnetic particle can be functionalized. The functionalization can include at least one of a molecular recognition moiety, an optical tag, an acidic moiety, a basic moiety, a cationic moiety, and anionic moiety, a hydrophilic moiety, a hydrophobic moiety, or a stimulus-responsive molecule or a mixture thereof.

The method can also include positioning an additional fluid bead in contact with the superhydrophobic surface, varying the magnetic field intensity so as to move the magnetically active fluid bead toward the additional fluid bead, and contacting the magnetically active fluid bead with the additional fluid bead with a force sufficient to overcome surface tension of the magnetically active fluid bead or the additional fluid bead, thereby coalescing the two beads. The additional fluid bead can include an additional coated magnetically active substrate and an additional fluid drop formed around the additional coated substrate. The additional coated magnetically active substrate can be coated with an anti-fouling layer. The additional coated magnetically active substrate can be coated with polysiloxane. The additional fluid bead can include at least one of a biologically active agent or a pharmaceutically active agent or a mixture thereof. The additional magnetically active substrate can include a paramagnetic particle. The paramagnetic particle can be functionalized. The functionalization can include at least one of a molecular recognition moiety, an optical tag, an acidic moiety, a basic moiety, a cationic moiety, and anionic moiety, a hydrophilic moiety, a hydrophobic moiety, or a stimulus-responsive molecule or a mixture thereof.

In another aspect, a method of immobilizing a fluid bead on a surface can include positioning a magnetically active fluid bead, comprising a magnetically active substrate enclosed by a coating and a fluid drop formed around the coated substrate, in contact with a superhydrophobic surface and coupling a stationary magnetic field with at least a portion of the bead.

This and other aspects can include one or more of the following features. The coating can be an anti-fouling layer. The coating can include polysiloxane.

In another aspect, a method of immobilizing a fluid bead on a surface can include positioning a fluid bead in contact with a surface having a more hydrophobic region and a less hydrophobic region and contacting the bead with the less hydrophobic region.

This and other aspects can include one or more of the following features. The more hydrophobic surface can be a superhydrophobic surface. The fluid bead can include a magnetically active substrate enclosed by a coating and a fluid drop formed around the coated substrate. The coating can be an anti-fouling layer. The coating can be polysiloxane.

In another aspect, a method of dispensing a magnetically active fluid bead can include positioning a fluid, comprising a suspension of coated, magnetically active particles, within a reservoir having an opening, and increasing the pressure within the reservoir, thereby dispensing a bead of fluid, containing a coated, magnetically active particle, through the opening.

This and other aspects can include one or more of the following features. The coating can be an anti-fouling layer. The coating can include polysiloxane.

In another aspect, a method of dispensing a fluid bead can include positioning a fluid, comprising a suspension of coated, magnetically active particles, within a reservoir having an opening, coupling a magnetic field with at least a portion of the fluid, and moving the magnetic field away from the reservoir, thereby dispensing a bead of the fluid, containing a coated, magnetically active particle, through the opening.

This and other aspects can include one or more of the following features. The reservoir can include a substantially enclosed chamber. The coating can be an anti-fouling layer. The coating can include polysiloxane.

In another aspect, a method of dividing a fluid bead can include positioning a magnetically active fluid bead, comprising a magnetically active substrate enclosed by a coating and a fluid drop formed around the coated substrate, in contact with a superhydrophobic surface, coupling a first magnetic field with at least a first portion of the fluid bead, coupling a second magnetic field with at least a second portion of the fluid bead, and varying the first magnetic field intensity so as to move the first portion substantially away from the second magnetic field with a force sufficient to overcome surface tension of the magnetically active fluid bead, thereby dividing the first portion of the bead from the second portion of the bead.

This and other aspects can include one or more of the following features. The coating can be an anti-fouling layer. The coating can include polysiloxane.

In another aspect, a digital isoelectric focusing method can include providing a magnetically active fluid bead, positioning the bead in contact with a superhydrophobic surface, coupling an electric field with the bead, thereby generating a pH gradient within the bead, allowing the first protein to migrate along the pH gradient to the first isoelectric point, allowing the second protein to migrate along the pH gradient to the second isoelectric point, coupling a first magnetic field with at least a first portion of the bead, wherein the first portion comprises the first isoelectric point, coupling a second magnetic field with at least a second portion of the bead wherein the second portion comprises the second isoelectric point, varying the first magnetic field intensity so as to move the first portion substantially away from the second magnetic field with a force sufficient to overcome surface tension of the magnetically active fluid bead, thereby dividing the first portion of the bead from the second portion of the bead.

The magnetically active fluid bead includes a magnetically active substrate enclosed by a coating, a fluid drop formed around the coated substrate, ampholytes, a first protein having a first isoelectric point, and a second protein having a second isoelectric point different from the first isoelectric point,

This and other aspects can include one or more of the following features. The providing act can be performed before the electric field is coupled with the bead. The coating can be an anti-fouling layer. The coating can include a polysiloxane.

In another aspect, a magnetically active fluid bead can include a magnetically active substrate enclosed by a coating and a fluid drop formed around the coated substrate.

This and other aspects can include one or more of the following features. The coating can be an anti-fouling layer. The coating can include polysiloxane. The fluid drop can include an aqueous fluid. The aqueous fluid can include at least one of water, sea water, saliva, blood, semen, plasma, urine, lymph, serum, tears, vaginal fluid, sweat, plant or vegetable extract fluid, or cell or tissue culture media, or a mixture thereof. The fluid drop can include at least one of a biologically active agent or a pharmaceutically active agent or a mixture thereof. The de fluid drop can include ampholytes. The fluid drop can include at least one of a chemically active agent, a chemical labeling agent, or a radioactive agent or a mixture thereof. The magnetically active substrate can include at least one of a paramagnetic material, a diamagnetic material, or a ferromagnetic material or a mixture thereof. The at least one of a paramagnetic material, a diamagnetic material, or a ferromagnetic material or a mixture thereof can be present in the fluid bead at a concentration of from about 0.05% (w/v) to about 5% (w/v), from about 0.1% (w/v) to about 10% (w/v), from about 0.5% (w/v) to about 5% (w/v), from about 1% (w/v) to about 10% (w/v), or from about 0.1% (w/v) to about 1% (w/v). The magnetically active substrate can be a paramagnetic particle. The paramagnetic particle can be functionalized. The functionalization can include at least one of a molecular recognition moiety, an optical tag, an acidic moiety, a basic moiety, a cationic moiety, and anionic moiety, a hydrophilic moiety, a hydrophobic moiety, or a stimulus-responsive molecule or a mixture thereof. The magnetically active substrate can include at least one of iron, nickel, or cobalt or a mixture thereof. The magnetically active substrate can include at least one paramagnetic carbonyl iron particle.

What is claimed is:

1. A magnetofluidic device comprising:
a superhydrophobic surface; and
a fluid sample in physical contact with the superhydrophobic surface, the fluid sample comprising a collection of particles coated with a passivating layer,
wherein the particles are magnetically active in that they respond to an applied magnetic field.

2. The magnetofluidic device of claim 1, wherein the passivating layer comprises a silicon-oxygen backbone.

3. The magnetofluidic device of claim 2, wherein the passivating layer comprises a polysiloxane.

4. The magnetofluidic device of claim 1, wherein the passivating layer comprises a polymerization product of tetraorthosilicate.

5. The magnetofluidic device of claim 1, wherein the magnetofluidic device comprises a digital magnetofluidic device.

6. The magnetofluidic device of claim 1, wherein the fluid sample comprises an aqueous solution.

7. The magnetofluidic device of claim 1, wherein the fluid sample comprises a bodily fluid.

8. The magnetofluidic device of claim 1, wherein the fluid sample comprises a solution of a biologically active agent, a pharmaceutically active agent, or a mixture thereof.

9. The magnetofluidic device of claim 1, wherein the collection of particles comprises a collection of paramagnetic particles.

10. The magnetofluidic device of claim 1, wherein the superhydrophobic surface comprises a nanostrutured surface.

11. The magnetofluidic device of claim 1, further comprising a device to apply a magnetic field to the fluid sample, wherein the applied magnetic field is of sufficient strength to induce movement of the fluid sample.

12. The magnetofluidic device of claim 1, wherein the fluid sample comprises a fluid droplet.

13. The magnetofluidic device of claim 1, wherein the passivating layer comprises a hydrophilic surface.

14. The magnetofluidic device of claim 1, wherein the passivating layer comprises a barrier to oxidation of the magnetically active particles.

15. The magnetofluidic device of claim 1, wherein the passivating layer comprises a barrier to reaction of the magnetically active particles with the fluid sample.

16. The magnetofluidic device of claim 1, wherein the fluid sample rests on the superhydrophobic surface.

17. A method comprising:
applying a magnetic field to a fluid sample that is in contact with a superhydrophobic surface, the fluid sample comprising a collection of particles coated with a passivating layer, wherein
the particles are magnetically active in that they respond to the applied magnetic field; and
the response of the magnetically active particles either induces or prevents movement of the fluid sample.

18. The method of claim 17, wherein applying the magnetic field comprises moving a source of the magnetic field to induce corresponding movement of the fluid sample.

19. The method of claim 17, wherein applying the magnetic field comprises changing a magnitude of the applied magnetic field to induce movement of the fluid sample.

20. The method of claim 17, wherein applying the magnetic field comprises moving the fluid sample into contact with a second fluid sample.

21. The method of claim 20, wherein the second fluid sample is pinned at a defect in the superhydrophobic surface.

22. The method of claim 20, wherein the second fluid sample does not include magnetically active particles.

23. The method of claim 20, further comprising moving a combination of the fluid sample and the second fluid sample.

24. The method of claim 17, wherein applying the magnetic field comprises splitting the fluid sample into a first aliquot and a second aliquot.

25. The method of claim 24, wherein splitting the fluid sample comprises applying an electric field to the fluid sample.

26. The method of claim 25, wherein applying the electric field comprises isoelectric focusing of a biopolymer in the fluid sample.

27. A system comprising:
a superhydrophobic surface;
a magnetic field source disposed to apply a magnetic field across the superhydrophobic surface; and
a fluid sample in physical contact with the superhydrophobic surface, the fluid sample comprising a collection of particles coated with a passivating layer,
wherein the particles are magnetically active in that they respond to the applied magnetic field.

28. The system of claim 27, further comprising a controller to control a magnitude or a direction of the magnetic field.

29. The system of claim 28, wherein the controller is configured to change the magnetic field generated by an electromagnet.
